(19) United States
(12) Patent Application Publication
(10) Pub. No.: US 2026/0283590 A1
(43) Pub. Date: Sep. 24, 2026

(54) THREE-DIMENSIONAL CHARACTERIZATION OF ATHEROSCLEROTIC PLAQUES USING TWO-DIMENSIONAL ULTRASOUND IMAGES

(71) Applicant: SONARO INC., Montréal, Québec (CA)

(72) Inventors: Karina GASBARRINO, Montréal, Québec (CA); Kashif KHAN, Montréal, Québec (CA); Alex BENJAMIN, Montréal, Québec (CA); Thierry BEIKO, Montréal, Québec (CA)

(73) Assignee: SONARO INC., Montréal, Québec (CA)

(21) Appl. No.: 19/474,596

(22) PCT Filed: Apr. 15, 2024
(86) PCT No.: PCT/CA2024/050484

§ 371 Date: Oct. 10, 2025

Related U.S. Application Data
(60) Provisional application No. 63/495,935, filed on Apr. 13, 2023.

(51) Int. Cl. A61B 8/08; A61B 8/00; G06T 7/00; G06T 7/11; G06T 7/62; G16H 50/30

(52) U.S. Cl. CPC A61B 8/0891; A61B 8/4254; A61B 8/4263; A61B 8/5223; A61B 8/5292; G06T 7/0012; G06T 7/11; G06T 7/62; G16H 50/30; G06T 2207/10016; G06T 2207/10132; G06T 2207/20081; G06T 2207/20084; G06T 2207/30048; G06T 2207/30104

(57) ABSTRACT

Devices, systems, computer readable mediums and methods i) for computing a three-dimensional representation of the atherosclerotic plaque in at least one blood vessel from a plurality of two-dimensional ultrasound images, ii) for rapid acquisition of vascular imaging data; iii) for improving medical scan quality utilizing a guidance and feedback system; iv) for processing a three-dimensional reconstruction of a vasculature to simulate and predict flow dynamics in the vasculature; v) for setting boundary conditions and solving the Navier-Stokes equation for simulating blood flow in a vasculature; and/or for automated measurement of diameter and characterization of stenosis and disease from vascular structures within a body.

### CROSS-REFERENCE TO RELATED APPLICATION

This non-provisional patent application claims priority based upon the prior U.S provisional patent application entitled “METHOD AND SYSTEM FOR DETECTION AND THREE-DIMENSIONAL CHARACTERIZATION OF ATHEROSCLEROTIC PLAQUES USING TWO-DIMENSIONAL ULTRASOUND IMAGES”, application No. 63/495,935, filed 2023 Apr. 13, in the name of SONARO INC., which is hereby incorporated by reference in its entirety.

### TECHNICAL FIELD

The present disclosure relates to atherosclerotic plaque, and more particularly to atherosclerotic plaque detection using ultrasound.

### BACKGROUND

Atherosclerosis is one of the major causes of death and disability in the world today. It is an inflammatory process characterized by the buildup of fatty deposits that form in the arteries of the body. Atherosclerotic plaques mainly form around arterial bifurcations due to the high turbulent blood flow. These areas include the carotid, coronary, aortic, renal, femoral, popliteal, and other peripheral arteries. Plaques that form in each of these areas present their own unique thromboembolic risk. For example, plaques forming in the carotid arteries of the neck is a major cause of cerebrovascular ischemic events, such as a transient ischemic attack, stroke or amaurosis fugax, leading to high morbidity and mortality worldwide. Other plaques that form in the peripheral arteries can lead to bowel ischemia or leg amputation without intervention.

Current clinical practice includes the use of 2D B-mode ultrasound imaging as a first line imaging tool to detect atherosclerotic plaques and the extent of arterial stenosis to determine thromboembolic risk. It is also used to determine the management of patients towards pharmacotherapy or surgical intervention, depending on the severity of arterial stenosis and the symptomatic status of the patient.

Although ultrasound is the predominant imaging modality used to detect atherosclerotic plaques, it faces several challenges. First, the quality of the images is based strongly on the skillset and experience of the user. Second, the angle of the probe limits the interpretability of the images, leading to either over-or underestimation of arterial stenosis and misclassifying patient cardiovascular risk. Finally, clinical judgments are currently based on 2D measurements, however plaques are complex 3D structures with varying morphologies. An objective 3D analysis of plaques will provide a more accurate measurement of arterial stenosis and cardiovascular risk.

Ultrasound probes that produce 3D images are emerging at a rapid pace, but presently remain a minority in clinical and commercial settings. The primary drawbacks of inherently 3D probes are that they are expensive, not portable (need for computational resources), have a relatively small field of view and continue to be restricted to very high-end applications. Conventional ultrasound probes, on the other hand, are comparatively cheap, highly portable and have comparatively low computational needs. This makes it an ideal clinical tool for emergency, point of care, bedside or resource constrained settings.

It is well understood that ultrasound images are of a lower resolution than CT or MRI and are very dependent on the operator's experience and acquisition conditions. These limitations lead to high interobserver variability, which can have important clinical consequences such as low-quality images, misdiagnoses, and poor access to care due to varying expertise.

### SUMMARY

To address the limitations relating to ultrasound, the present disclosure describes a stitching together of 2D ultrasound images to create an accurate 3D representation of the blood vessel and underlying plaque in order to detect and characterize atherosclerotic plaques. This method and this system allow for accurate measurements of the 3D vessel wall and plaque in real-time for a faster and more accurate detection and characterization of the atherosclerotic plaque compared to conventional methods.

In a first aspect, the present disclosure relates to devices, systems, computer readable mediums and methods for computing a three-dimensional representation of the atherosclerotic plaque in at least one blood vessel from a plurality of two-dimensional ultrasound images.

In a second aspect, the present disclosure relates to devices, systems, computer readable mediums and methods for rapid acquisition of vascular imaging data.

In a third aspect, the present disclosure relates to devices, systems, computer readable mediums and methods for improving medical scan quality utilizing a guidance and feedback system.

In a fourth aspect, the present disclosure relates to devices, systems, computer readable mediums and methods for processing a three-dimensional reconstruction of a vasculature to simulate and predict flow dynamics in the vasculature.

In a fifth aspect, the present disclosure relates to devices, systems, computer readable mediums and methods for setting boundary conditions and solving the Navier-Stokes equation for simulating blood flow in a vasculature.

In a sixth aspect, the present disclosure relates to devices, systems, computer readable mediums and methods for automated measurement of diameter and characterization of stenosis and disease from vascular structures within a body.

In accordance with the first aspect, a method of characterizing an atherosclerotic plaque in vasculature is provided. The method comprises acquiring a plurality of two-dimensional ultrasound images of the vasculature using an ultrasound probe, each image being associated to positional data of the ultrasound probe relative to the vasculature at the time of image acquisition. The method also comprises segmenting the plurality of two-dimensional ultrasound images considering varying acoustic signatures between one or more plaque atherosclerotic bodies and vasculature tissue and computing a location value and an orientation value over time from positional data contained in more than one of the plurality of two-dimensional ultrasound images. The method also comprises computing a three-dimensional representation of the atherosclerotic plaque in at least one blood vessel of the vasculature from the location value, the orientation value and the segmented plurality of two-dimensional ultrasound images. In accordance with the first aspect, a non-transitory computer-readable medium may be provided that includes one or more instructions that, when executed by one or more processors of a device, cause the device to execute the method described hereinabove. Other embodiments of this aspect include corresponding computer systems, apparatus, and computer programs recorded on one or more computer storage devices, each configured to perform the actions of the methods.

In accordance with the first aspect, a device for characterizing an atherosclerotic plaque in vasculature comprises one or more processors configured to acquire a plurality of two-dimensional ultrasound images of the vasculature using an ultrasound probe, each image being associated to positional data of the ultrasound probe relative to the vasculature at the time of image acquisition; segment the plurality of two-dimensional ultrasound images considering varying acoustic signatures between one or more plaque atherosclerotic bodies and vasculature tissue; compute a location value and an orientation value over time from positional data contained in more than one of the plurality of two-dimensional ultrasound images; and compute a three-dimensional representation of the atherosclerotic plaque in at least one blood vessel of the vasculature from the location value, the orientation value and the segmented plurality of two-dimensional ultrasound images. Other embodiments of this aspect include corresponding computer systems, apparatus, and computer programs recorded on one or more computer storage devices, each configured to perform the actions of the methods.

In accordance with the first aspect, a system comprises an ultrasound probe that acquires a plurality of two-dimensional ultrasound images of the vasculature and one or more processors configured to associate positional data to a set of location-enhanced images of the plurality of two-dimensional ultrasound images relative to the vasculature at the time of image acquisition; segment the location-enhanced images considering varying acoustic signatures between one or more plaque atherosclerotic bodies and vasculature tissue; compute a location value and an orientation value over time from positional data contained in more than one of the location-enhanced images; and compute a three-dimensional representation of the atherosclerotic plaque in at least one blood vessel of the vasculature from the location value, the orientation value and the segmented location-enhanced images. Other embodiments of this aspect include corresponding computer systems, apparatus, and computer programs recorded on one or more computer storage devices, each configured to perform the actions of the methods.

Implementations in accordance with the first aspect, may include one or more of the following features. The positional data may include position sensors data obtained from position sensors of the ultrasound probe (e.g., associated to the ultrasound probe and/or thereon) and computing the location value and the orientation value is performed considering the position sensors data.

Optionally, additionally or alternatively, defining a three-dimensional space may be performed by analyzing images taken from one or more cameras associated to the ultrasound probe and computing the positional data for each image of the plurality of ultrasound images considering movement of the ultrasound probe relative to the three-dimensional space.

Optionally, additionally or alternatively, segmenting the plurality of two-dimensional ultrasound images may be performed using an artificial intelligence model trained using a dataset of preexisting images or videos segmented to distinguish plaque from surrounding vasculature tissue.

Optionally, additionally or alternatively, the plurality of two-dimensional ultrasound images may be B-mode ultrasound images that are in a DICOM format.

Optionally, additionally or alternatively, computing the location and the orientation may be performed using triangulation.

Optionally, additionally or alternatively, comprise the vasculature may be an artery, and the plurality of two-dimensional ultrasound images may depict a length of the artery.

Optionally, additionally or alternatively, comprise the plurality of two-dimensional ultrasound images may include depictions of cross-sections of the vasculature.

Optionally, additionally or alternatively, computing a three-dimensional reconstruction of the vasculature having the three-dimensional representation of the atherosclerotic plaque may be performed.

Optionally, additionally or alternatively, determining, from the three-dimensional reconstruction of the vasculature, one or more of: a diameter of the vasculature; an area of the vasculature; a volume of the vasculature; an intimal-medial thickness of the vasculature; and a stenosis of the vasculature may be performed.

Optionally, additionally or alternatively, generating the positional data may be performed by computing a set of spatial coordinates of an electromagnetic sensor of the ultrasonic probe (e.g., associated to the ultrasonic probe and/or thereon) considering location of an electromagnetic source and at least two angular values, a timestamp being used to associate the positional data to the plurality of two-dimensional ultrasound images.

Optionally, additionally or alternatively, calibrating electromagnetic sensor of the ultrasonic probe e.g., associated to the ultrasonic probe and/or thereon) considering the location of the electromagnetic source may be performed.

Optionally, additionally or alternatively, defining, from the three-dimensional representation of the atherosclerotic plaque, one or more of: an area and size of the atherosclerotic plaque; a volume of the atherosclerotic plaque; stenosis caused by the atherosclerotic plaque; echogenicity of the atherosclerotic plaque; composition of the atherosclerotic plaque; pixel density; and blood flow dynamics and characteristics may be performed.

Optionally, additionally or alternatively, computing a risk score of vascular thromboembolic event for a subject of the two-dimensional ultrasound images, the risk being defined based at least on: the three-dimensional representation of the atherosclerotic plaque and the three-dimensional reconstruction of the vasculature; plaque metrics computed from the three-dimensional reconstruction of the atherosclerotic plaque including one or more of the area and size of the atherosclerotic plaque, the volume of the atherosclerotic plaque, the stenosis caused by the atherosclerotic plaque, the echogenicity of the atherosclerotic, the plaque composition of the atherosclerotic plaque, the pixel density and the blood flow dynamics and characteristics; and one or more of age, gender, biological sex, weight, body mass index, ethnicity, history of smoking, cholesterol level, glucose level, and history of cardiovascular events of the subject may be performed.

Optionally, additionally or alternatively, defining the risk of vascular disease for the subject may be performed further considering published and validated thromboembolic risk score calculators, and where computing the risk score may include weighing the plaque characteristics and the patient data according to contribution to thromboembolic risk score calculators.

Optionally, additionally or alternatively, computing the risk score may be performed by further utilizing a trained artificial intelligence model configured to integrate the plaque characteristics and the patient data to output the risk score.

Optionally, additionally or alternatively, computing the risk score may be performed further considering additional clinical data including the subject's medical history related to one or more of hospitalizations, presence of transient ischemic attack, amaurosis fugax events, and stroke events.

Optionally, additionally or alternatively, computing the risk score may be performed further considering multiple reconstructed three-dimensional atherosclerotic plaques within the vasculature to calculate a comprehensive risk profile for the subject.

Optionally, additionally or alternatively, estimating a level of uncertainty associated with the computed location value and the orientation value and prompting a user to perform one or more scans of the vasculature using the ultrasound probe to obtain an additional set of two-dimensional ultrasound images may be performed. Implementations of the described techniques may include hardware, a method or process, or a computer tangible medium.

In accordance with the second aspect, a method for rapid acquisition of vascular imaging data is provided comprising initiating an imaging sequence by positioning an ultrasound probe at a predetermined starting location on the transverse plane of a target vasculature, a position-tracking sensor being attached to the ultrasound probe. The method also includes acquiring a plurality of two-dimensional ultrasound images of the vasculature using the ultrasound probe, each image containing positional data of the ultrasound probe relative to the vasculature at the time of image acquisition. The acquiring is performed in a single sweep along the artery in a duration substantially shorter than traditional methods, thereby reducing an amount of time required for an imaging session to less than 10 minutes, preferably less than 5 minutes. In accordance with the second aspect, a non-transitory computer-readable medium may be provided that includes one or more instructions that, when executed by one or more processors of a device, cause the device to execute the method described hereinabove. Other embodiments of this aspect include corresponding computer systems, apparatus, and computer programs recorded on one or more computer storage devices, each configured to perform the actions of the methods.

In accordance with the second aspect, a device is provided for rapid acquisition of vascular imaging data. The device comprises one or more processors configured to: initiate an imaging sequence by positioning an ultrasound probe at a predetermined starting location on the transverse plane of a target vasculature, a position-tracking sensor being attached to the ultrasound probe; and acquire a plurality of two-dimensional ultrasound images of the vasculature using the ultrasound probe, each image containing positional data of the ultrasound probe relative to the vasculature at the time of image acquisition. The acquiring is performed in a single sweep along the artery in a duration substantially shorter than traditional methods, thereby reduce an amount of time required for an imaging session to less than 10 minutes, preferably less than 5 minutes. Other embodiments of this aspect include corresponding computer systems, apparatus, and computer programs recorded on one or more computer storage devices, each configured to perform the actions of the methods.

In accordance with the second aspect, a system may include an ultrasound probe; a position-tracking sensor attached to the ultrasound probe; and one or more processors configured to: indicate, to an operator of the ultrasound probe, a starting location of the ultrasound probe at predetermined on the transverse plane of a target vasculature; indicate, to the operator, to perform a single sweep along the artery in a duration substantially shorter than traditional methods; receive a plurality of two-dimensional ultrasound images of the vasculature using the ultrasound probe, each image containing positional data of the ultrasound probe relative to the vasculature at the time of image acquisition. Other embodiments of this aspect include corresponding computer systems, apparatus, and computer programs recorded on one or more computer storage devices, each configured to perform the actions of the methods.

Implementations may include one or more of the following features. Optionally, performing one or more additional sweeps may be performed, each having a subsequent duration substantially shorter than the traditional methods; compiling a set of two-dimensional ultrasound images and positional data from the single sweep and the additional sweeps may be performed; and computing a three-dimensional representation of the atherosclerotic plaque in the vasculature from the set of two-dimensional ultrasound images and positional data may be performed.

Optionally, additionally or alternatively, synchronizing and processing the plurality of two-dimensional ultrasound images and the positional data thereby facilitating computation of a three-dimensional vascular model may be performed in real-time or quasi-real-time.

Optionally, additionally or alternatively, the duration of the sweep may be set based on the length and complexity of the target vasculature. Implementations of the described techniques may include hardware, a method or process, or a computer tangible medium.

In accordance with the third aspect, a method for improving medical scan quality utilizing a guidance and feedback system is provided. The method comprises identifying one or more parameters affecting scan quality, the one or more parameters having artery location, ultrasound image gain, contrast, or brightness, and image quality metrics; and providing operator feedback, the feedback having step-by-step instructions for corrective actions to enhance scan quality. In accordance with the third aspect, a non-transitory computer-readable medium may be provided that includes one or more instructions that, when executed by one or more processors of a device, cause the device to execute the method described hereinabove. Other embodiments of this aspect include corresponding computer systems, apparatus, and computer programs recorded on one or more computer storage devices, each configured to perform the actions of the methods.

In accordance with the third aspect, a device is provided for improving medical scan quality utilizing a guidance and feedback system. The device includes one or more processors configured to: identify one or more parameters affecting scan quality, the one or more parameters having artery location, ultrasound image gain, contrast, or brightness, and image quality metrics; and provide operator feedback, the feedback having step-by-step instructions for corrective actions to enhance scan quality. Other embodiments of this aspect include corresponding computer systems, apparatus, and computer programs recorded on one or more computer storage devices, each configured to perform the actions of the methods.

In accordance with the third aspect, a system comprises one or more processors configured to identify one or more parameters affecting scan quality, the one or more parameters having artery location, ultrasound image gain, contrast, or brightness, and image quality metrics and provide operator feedback, the feedback having step-by-step instructions for corrective actions to enhance scan quality. Other embodiments of this aspect include corresponding computer systems, apparatus, and computer programs recorded on one or more computer storage devices, each configured to perform the actions of the methods.

Implementations of the third aspect may include one or more of the following features. Optionally, the operator feedback may be delivered in real-time or quasi real-time through visual indicators, audio signals, haptic signals and/or textual instructions thereby facilitating comprehension and intervention of the operator during scanning procedures.

Optionally, additionally or alternatively, an amplitude of the operator feedback may be proportional to an amplitude of a deviation between an expected value of the one or more parameters and an actual value for the one or more parameters. Implementations of the described techniques may include hardware, a method or process, or a computer tangible medium.

In accordance with the fourth aspect, a method for processing a three-dimensional reconstruction of a vasculature to simulate and predict flow dynamics in the vasculature is provided. The method comprises receiving a three-dimensional structure representing the vasculature, analyzing the three-dimensional structure using a defect elimination algorithm to selectively fill gaps and correct anomalies and enhancing the three-dimensional structure using mesh refinement techniques to provide smooth and accurate representation of a geometry of the vasculature. In accordance with the fourth aspect, a non-transitory computer-readable medium may be provided that includes one or more instructions that, when executed by one or more processors of a device, cause the device to execute the method described hereinabove. Other embodiments of this aspect include corresponding computer systems, apparatus, and computer programs recorded on one or more computer storage devices, each configured to perform the actions of the methods.

In accordance with the fourth aspect, a device is provided for processing a three-dimensional reconstruction of a vasculature to simulate and predict flow dynamics in the vasculature. The device comprises one or more processors configured to: receive a three-dimensional structure representing the vasculature; analyze the three-dimensional structure using a defect elimination algorithm to selectively fill gaps and correct anomalies; and enhance the three-dimensional structure using mesh refinement techniques to provide smooth and accurate representation of a geometry of the vasculature. Other embodiments of this aspect include corresponding computer systems, apparatus, and computer programs recorded on one or more computer storage devices, each configured to perform the actions of the methods.

In accordance with the fourth aspect, a system is provided for processing a three-dimensional reconstruction of a vasculature to simulate and predict flow dynamics in the vasculature. The system comprises one or more processors configured to: receive a three-dimensional structure representing the vasculature; analyze the three-dimensional structure using a defect elimination algorithm to selectively fill gaps and correct anomalies; and enhance the three-dimensional structure using mesh refinement techniques to provide smooth and accurate representation of a geometry of the vasculature.

In accordance with the fifth aspect, a method for setting boundary conditions and solving the Navier-Stokes equation for simulating blood flow in a vasculature is provided. The method comprises determining time-varying pressure conditions at an inlet based on an average systolic and diastolic blood pressures of a patient; estimating outlet boundary conditions employing numerical techniques thereby ensuring consistency with the simulation results; utilizing computational fluid dynamics (CFD) solvers to numerically solve the Navier-Stokes equation for non-Newtonian fluid flow within the vasculature; and generating a pressure distribution field output and a flow velocity profiles field output along the length of the vessel. In accordance with the fifth aspect, a non-transitory computer-readable medium may be provided that includes one or more instructions that, when executed by one or more processors of a device, cause the device to execute the method described hereinabove. Other embodiments of this aspect include corresponding computer systems, apparatus, and computer programs recorded on one or more computer storage devices, each configured to perform the actions of the methods.

In accordance with the fifth aspect, a device is provided for setting boundary conditions and solving the Navier-Stokes equation for simulating blood flow in a vasculature. The device comprises one or more processors configured to: determine time-varying pressure conditions at an inlet based on an average systolic and diastolic blood pressures of a patient; estimate outlet boundary conditions employing numerical techniques thereby ensuring consistency with the simulation results; utilize computational fluid dynamics (CFD) solvers to numerically solve the Navier-Stokes equation for non-Newtonian fluid flow within the vasculature; and generate a pressure distribution field output and a flow velocity profiles field output along the length of the vessel. Other embodiments of this aspect include corresponding computer systems, apparatus, and computer programs recorded on one or more computer storage devices, each configured to perform the actions of the methods.

In accordance with the fifth aspect, a system is provided for setting boundary conditions and solving the Navier-Stokes equation for simulating blood flow in a vasculature. The system comprises one or more processors configured to: determine time-varying pressure conditions at an inlet based on an average systolic and diastolic blood pressures of a patient; estimate outlet boundary conditions employing numerical techniques thereby ensuring consistency with the simulation results; utilize computational fluid dynamics (CFD) solvers to numerically solve the Navier-Stokes equation for non-Newtonian fluid flow within the vasculature; and generate a pressure distribution field output and a flow velocity profiles field output along the length of the vessel.

In accordance with the sixth aspect, a method for automated measurement of diameter and characterization of stenosis and disease from vascular structures within a body is provided. The method comprises receiving a three-dimensional structure representing a vasculature; removing noise and outliers from an input point cloud data corresponding to the three-dimensional structure; slicing the point cloud data perpendicular to a longitudinal axis of the vasculature to generate cross-sectional profiles. The method also comprises, from each of the cross-section profiles, determining cross-sectional diameters of the vasculature; computing statistical measures across at least a subset of the cross-sectional diameters, the statistical measures having one or more of mean, variance, minimum, maximum, and median diameters; and generating reports summarizing vascular structure characteristics. In accordance with the sixth aspect, a non-transitory computer-readable medium may be provided that includes one or more instructions that, when executed by one or more processors of a device, cause the device to execute the method described hereinabove. Other embodiments of this aspect include corresponding computer systems, apparatus, and computer programs recorded on one or more computer storage devices, each configured to perform the actions of the methods.

In accordance with the sixth aspect, a device is provided for automated measurement of diameter and characterization of stenosis and disease from vascular structures within a body. The device comprises one or more processors configured to: receive a three-dimensional structure representing a vasculature; remove noise and outliers from an input point cloud data correspond to the three-dimensional structure; slice the point cloud data perpendicular to a longitudinal axis of the vasculature to generate cross-sectional profiles; from each of the cross-section profiles, determine cross-sectional diameters of the vasculature; compute statistical measures across at least a subset of the cross-sectional diameters, the statistical measures having one or more of mean, variance, minimum, maximum, and median diameters; and generate reports summarizing vascular structure characteristics. Other embodiments of this aspect include corresponding computer systems, apparatus, and computer programs recorded on one or more computer storage devices, each configured to perform the actions of the methods.

In accordance with the sixth aspect, a system is provided for automated measurement of diameter and characterization of stenosis and disease from vascular structures within a body. The system comprises one or more processors configured to: receive a three-dimensional structure representing a vasculature; remove noise and outliers from an input point cloud data corresponding to the three-dimensional structure; slice the point cloud data perpendicular to a longitudinal axis of the vasculature to generate cross-sectional profiles; from each of the cross-section profiles, determine cross-sectional diameters of the vasculature; compute statistical measures across at least of subset of the cross-sectional diameters, the statistical measures comprising one or more of mean, variance, minimum, maximum, and median diameters; and generate reports summarizing vascular structure characteristics.

Implementations of the sixth aspect may include one or more of the following features. Optionally, the three-dimensional structure is represented in a file format of one or more of STL, OBJ, PLY, and IDS. Implementations of the described techniques may include hardware, a method or process, or a computer tangible medium.

### BRIEF DESCRIPTION OF THE DRAWINGS

The invention will be better understood by way of the following detailed description of embodiments of the invention with reference to the appended drawings, in which:

FIG. 1 is a block diagram of an exemplary system for generating a three-dimensional reconstruction of an atherosclerotic plaque from a plurality of two-dimensional ultrasound images;

FIGS. 2A, 2B, 2C, 2D and 2E are exemplary images with FIG. 2 A presenting an exemplary B-mode ultrasound image of a cross-section of vasculature with a plaque and FIGS. 2B, 2C, 2D and 2E depicting a blood vessel with a 3d representation of plaque;

FIG. 3 is a diagram representing an exemplary deep convolutional neural network for atherosclerotic plaque detection;

FIG. 4 is a visual representation of cross-sectional two-dimensional ultrasound images generated by an ultrasound probe, leading to a three-dimensional reconstruction of the atherosclerotic plaque;

FIG. 5 is a block diagram of exemplary software architecture for generating a three-dimensional reconstruction of an atherosclerotic plaque from two-dimensional ultrasound images;

FIG. 6 is a flowchart diagram of an exemplary method for generating a three-dimensional reconstruction atherosclerotic plaque from two-dimensional ultrasound images;

FIG. 7 is a flowchart diagram of an exemplary method for computing a three-dimensional representation atherosclerotic plaque from two-dimensional ultrasound images;

FIG. 8 is a flowchart diagram of an exemplary method for computing a three-dimensional reconstruction atherosclerotic plaque from two-dimensional ultrasound images;

FIG. 9 is a flowchart diagram of an exemplary method for providing operator feedback during acquisition of two-dimensional ultrasound images;

FIG. 10 is a flowchart diagram of an exemplary method for generating pressure distribution profiles related to atherosclerotic plaque; and

FIG. 11 is a flowchart diagram of an exemplary method for characterization of a vascular structure from two-dimensional ultrasound images.

### DETAILED DESCRIPTION

In a first aspect, the present disclosure relates to devices, systems, computer readable mediums and methods for computing a three-dimensional representation of the atherosclerotic plaque in at least one blood vessel from a plurality of two-dimensional ultrasound images.

In a second aspect, the present disclosure relates to devices, systems, computer readable mediums and methods for rapid acquisition of vascular imaging data.

In a third aspect, the present disclosure relates to devices, systems, computer readable mediums and methods for improving medical scan quality utilizing a guidance and feedback system.

In a fourth aspect, the present disclosure relates to devices, systems, computer readable mediums and methods for processing a three-dimensional reconstruction of a vasculature to simulate and predict flow dynamics in the vasculature.

In a fifth aspect, the present disclosure relates to devices, systems, computer readable mediums and methods for setting boundary conditions and solving the Navier-Stokes equation for simulating blood flow in a vasculature.

In a sixth aspect, the present disclosure relates to devices, systems, computer readable mediums and methods for automated measurement of diameter and characterization of stenosis and disease from vascular structures within a body.

Synergy exists between the different aspects disclosed herein and the different aspects may be used in various combinations to provide various advantages that may not be achievable by using only a subset or only one of the different aspects. However, skilled persons will readily recognize that each of the different aspects is also exploitable in isolation.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

From the foregoing it will be appreciated that, although specific embodiments have been described herein for purposes of illustration, various modifications may be made without deviating from the spirit and scope of the teachings. Accordingly, the claims are not limited by the disclosed embodiments.

In the present disclosure, by “thromboembolic event”, it is meant the result of the obstruction of a blood vessel by a blood clot that has become dislodged from another site in the circulatory system.

In the present disclosure, by “plaque body”, it is meant an atherosclerotic plaque that is captured on an image, such as a two-dimensional ultrasound image of vasculature.

In the present disclosure, by “positional data”, it is meant data that allows by direct reference or through one or more computing step, to determine one or more of a position, change in position, orientation, change of orientation of an ultrasound probe and/or one or more sensors related and/or linked thereto.

In the present disclosure, by “subject”, it is meant mammal and non-mammals. Mammals mean any member of the mammalia class including, but not limited to, humans. Non-mammals include birds, reptiles, etc. The term “subject” should not bring on any limitations as to the sex or age.

In the present disclosure, by “vasculature”, it is meant one or more blood vessels in a subject. Exemplary vasculature that may be captured using ultrasound in accordance with the present teachings are arteries and veins. Use of the term vasculature herein may refer to a single blood vessel, or to multiple blood vessels.

In the present disclosure, by “ultrasound probe”, it is meant a system that allows 2D ultrasonic images of a vasculature of a subject to be obtained. Ultrasonic probe and ultrasound probe may be used interchangeably. In general, ultrasound probes would have a resolution range of 0.02 mm to 2 mm, often between 0.1 mm and 1 mm. In the context of the present disclosure, quality of the 2D ultrasonic images in these ranges is appropriate.

In the present disclosure, by “real time” or “quasi real time”, it is meant the processing of data or the execution of actions in a manner that is either instant or approaches instantaneity, coinciding closely with their occurrence or acquisition. An imperceptible delay is defined from the viewpoint of the user or system, thereby guaranteeing that the information or results disseminated are contemporary, mirroring the current status or situation of the subject matter under observation or management. In the context of the present disclosure, 250 ms would, in most circumstances, still be considered coinciding from the perspective of an operator.

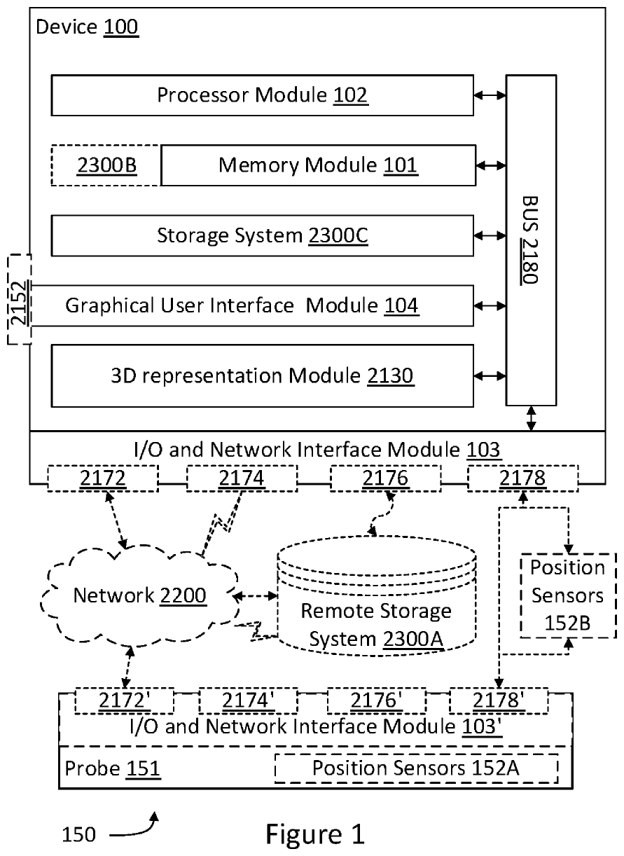

Reference is now made to the drawings in which FIG. 1 shows a logical modular representation of an exemplary system 150 comprising a device 100. The device 100 comprises a memory module 102, a processor module 101, a 3D representation module 2130 and a network interface module 2170. The device 100 may also include a user interface module 104.

The system 150 may comprise a storage system 2300 for storing and accessing long-term (i.e., non-transitory) data and may further log data while the device 100 is being used. FIG. 1 shows examples of the storage system 2300 as a distinct database system 2300A, a distinct module 2300C of the device 100 or a sub-module 2300B of the memory module 102 of the device 100. The storage system 2300 may be distributed over different systems A, B, C. The storage system 2300 may comprise one or more logical or physical as well as local or remote hard disk drive (HDD) (or an array thereof). The storage system 2300 may further comprise a local or remote database made accessible to the device 100 by a standardized or proprietary interface or via an input/output and network interface module 103.

The i/o and network interface module 103 represents at least one physical interface that can be used to communicate with other network nodes. The i/o and network interface module 103 may be made visible to the other modules of the device 100 through one or more logical interfaces. The actual stacks of protocols used by the physical network interface(s) and/or logical network interface(s) 2172-2178 of the i/o and network interface module 103 do not affect the teachings of the present invention.

The processor module 101 may represent a single processor with one or more processor cores or an array of processors, each comprising one or more processor cores. The memory module 102 may comprise various types of memory (different standardized or kinds of Random Access Memory (RAM) modules, memory cards, Read-Only Memory (ROM) modules, programmable ROM, etc.).

A bus 2180 is depicted as an example of means for exchanging data between the different modules of the device 100. The teachings presented herein are not affected by the way the different modules exchange information. For instance, the memory module 102 and the processor module 101 could be connected by a parallel bus, but could also be connected by a serial connection or involve an intermediate module (not shown) without affecting the teachings of the present invention.

A 3D representation module 2130 provides 3D representation-related services to the device 100, which will be described in more details hereinbelow.

The variants of processor module 101, memory module 102 and io/and network interface module 103 usable in the context of the present invention will be readily apparent to persons skilled in the art. Likewise, even though explicit mentions of the 3D representation module 2130, the memory module 102, the user interface module 2150 and/or the processor module 101 are not made throughout the description of the present examples, persons skilled in the art will readily recognize when such modules are used in conjunction with other modules of the device 100 to perform routine as well as innovative elements presented herein.

The system 150 an ultrasound probe 151 and one or more position sensors 152A and/152B associated and/or mounted on the ultrasound probe 151. Position sensors 152A and position sensors 152B are depicted on FIG. 1 for illustrative purpose and referred to herein as the one or more sensors 152. In some implementations, only the position sensors 152A or only the position sensors 152B are provided. For instance, the positions sensors 152B may be sufficient when positional data is obtained from analysis of images taken from one or more camera (not shown) depicting the ultrasound probe 151 in action (i.e., the positions sensors 152B therefore comprise the one or more camera). Conversely, the positions sensors 152A may be sufficient when positional data is obtained from a sensor device that is part of or fixed to the ultrasound probe 151. In some other implementations, the position sensors 152A and the position sensors 152B may be used concurrently and/or alternatively, e.g., as backup source of positional data, to validate and/or refine the positional data.

The i/o and network interface module 103 may be used for communication with the ultrasound probe 151 and/or the position sensors 152.

A separate I/O interface (not shown) may also be provided in the ultrasound probe 151 for communicating with the position sensor(s) 152.

The computer readable memory 102 may further store program instructions and data used by the processor 101. The computer readable memory 102 may also store two-dimensional ultrasound images and/or videos, positional data received from the position sensor(s) 152, Doppler images, etc. The memory 102 may also store information regarding data source(s) that are accessible by the device 100, such as the identity of the data sources. The memory 102 may be non-transitory.

In some examples, the computing device 100 may be in communication with the storage system 2300A for storing two-dimensional ultrasound images, three-dimensional reconstruction models of plaques and/or vasculature, etc. Alternatively or additionally, in some examples, the storage system 2300B or 2300C of the computing device 100 may have a local database that contains three-dimensional reconstruction models of plaques and/or vasculature, etc.

The graphical user interface module 104 may have a touchscreen or other input-enabled functionality 2152. The graphical user interface module may be configured to display the two-dimensional ultrasound images, the three-dimensional reconstructions of the plaques and/or vasculature, etc.

The computing device 100 may have (or be connect to) any suitable I/O device(s), for example, such as a keyboard, a mouse, a touchscreen etc.

The ultrasound probe 151 is suitable for generating two-dimensional ultrasound images of tissue of a subject. Exemplary two-dimensional ultrasound images may be of the following types: B-mode, M-mode, Doppler, shear wave elastography, etc.

The position sensor(s) 152 are configured to generate positional data of the ultrasound probe 151, the positional data containing or may be analyzed to determine one or more angles of the position of the ultrasound probe 151 when capturing a two-dimensional ultrasound image. The position sensor(s) 152 may include one or more RGB (red-blue-camera) cameras for capturing coloured images, one or more infrared cameras, one or more accelerometers, one or more gyroscopes, one or more magnetometers, etc. A mount may be provided to join the one or more position sensors 152 to the ultrasound probe 151. In some examples, the one or more position sensors 152 may be integrated to the ultrasound probe 151.

In some instances, the position sensors 152 may be fixed to other surfaces or objects in an area where the medical imaging using the ultrasound probe is performed, such as a wall or tablet when the position sensors 152 relies on optical or electromagnetic tracking.

In one example, the position sensors 152 may include one or more cameras, IMUs, optical trackers or electromagnetic trackers joined to the ultrasound probe 151 using a rigid mounting.

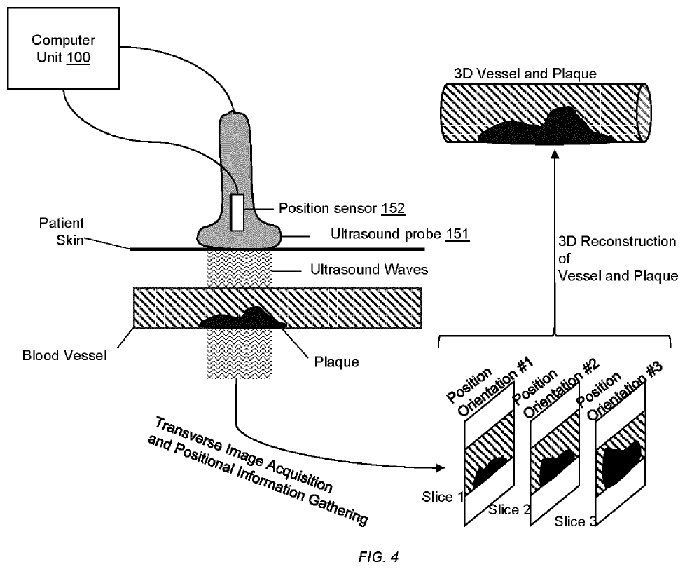

Reference is now made to FIG. 5, illustrating an exemplary software architecture 500 of program code located in memory 102 of the device 100 for generating a three-dimensional reconstruction of an atherosclerotic plaque.

The device 100 has program code, stored in memory 102, that includes a data collection module 510. The device 100 has program code, stored in memory 102, that includes a position generation module 520. The device 100 has program code, stored in memory 102, that includes an image segmentation module 530. The device 100 has program code, stored in memory 102, that includes a 3D reconstruction module 540. The device 100 may have program code, stored in memory 102, that includes a risk evaluation module 550. Each of the data collection module 510, position generation module 520, image segmentation module 530, 3D reconstruction module 540 and risk evaluation module 550 includes program code configured to implement the functionality of the modules as is described herein.

The data collection module 510 includes program code stored in memory 102 that, when executed by the processor 101, causes the processor 101 to receive data related to the ultrasound probe 151. More particularly, the processor 101 is caused to receive the two-dimensional ultrasound image information or two-dimensional ultrasound images from the ultrasound probe or an ultrasound machine with the ultrasound probe 151. For instance, the ultrasound information generated by use of the ultrasound probe 151 on the subject may be visualized using a separate ultrasound machine as two-dimensional ultrasound images, the two-dimensional ultrasound images then received by the processor 101 executing the code of the data collection module 510. In some embodiments, the data collection module 510 may include program code to cause the processor 101 to receive the ultrasound information generated by use of the ultrasound probe 151, the two-dimensional ultrasound images generated by the data collection module 510 from the ultrasound data produced from the ultrasound probe 151.

The data collection module 510 also causes the processor 101 to receive I from the one or more position sensors 152 joined to the ultrasound probe 151. The positional data may be generated and/or received continuously, when the ultrasound probe 151 is in use, or when the data collection module 510 also received a two-dimensional ultrasound image. In some examples, the two-dimensional ultrasound image may include a timestamp that may be correlated with the timestamp of the positional data to associate the appropriate positional data to its respective two-dimensional ultrasound image, the positional data indicative of the position of the ultrasound probe 151 when the data was generated by the ultrasound probe 151 leading to the generation of the two-dimensional ultrasound image.

In some instances, the plurality of two-dimensional ultrasound images may form an image stream.

The position generation module 520 includes program code stored in memory 102 that, when executed by the processor 101, causes the processor 101 to generate an orientation and a position of the ultrasound probe 151, from the positional data, when the ultrasound probe 151 was generating the ultrasound data corresponding to each of the plurality of two-dimensional ultrasound images. The position generation module 520 may cause the processor 101 to generate a position and an orientation of the ultrasound probe 151 at specific timestamps corresponding to when the two-dimensional ultrasound images are generated, or continuously when the two-dimensional ultrasound images form an image stream of two-dimensional ultrasound images.

The image segmentation module 530 includes program code stored in memory 102 that, when executed by the processor 101, causes the processor to perform image analysis of the plurality of two-dimensional ultrasound images and segments the plurality of two-dimensional ultrasound images by identifying the portions of the two-dimensional ultrasound image displaying an atherosclerotic plaque and portions of the image displaying surrounding vascular tissue. A boundary line may be added by the processor 101 executing the program code of the image segmentation module 530 to visually identify the outer perimeter of each of the atherosclerotic plaques appearing in the two-dimensional ultrasound image.

In some embodiments, the image segmentation module 530 includes a trained artificial intelligence model for performing image recognition, analysis and segmentation. The artificial intelligence model may be trained using a dataset of segmented ultrasound images, where the atherosclerotic plaque(s) appearing in the two-dimensional ultrasound image used for training have been identified (e.g., manually).

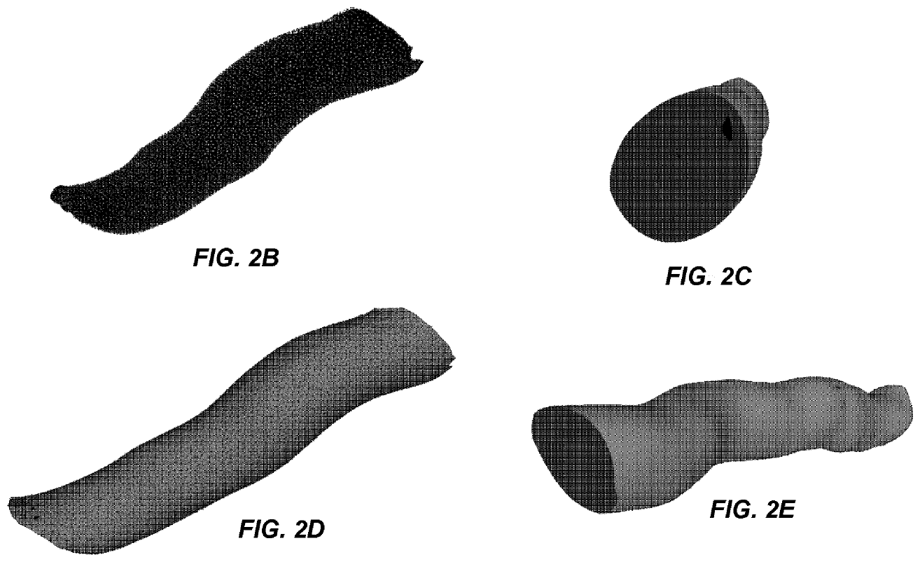

An exemplary illustration of an artificial intelligence model (e.g. a convolutional neural network) that is trained to perform image segmentation of two-dimensional ultrasound images of vasculature may be found at FIG. 3. The artificial intelligence model may be provided with a dataset of test images. Once the artificial intelligence outputs a segmentation of the testing ultrasound image, segmenting the image to identify the atherosclerotic plaque 310, the image that has been segmented by the artificial intelligence is compared to a prediction test image. Differences between the output image that has been segmented by the artificial intelligence model and the prediction test image are returned to the artificial intelligence model to further train the artificial intelligence model until a threshold level of accuracy has been attained by the trained artificial intelligence model.

The artificial intelligence model may be one as is known in the art for performing object recognition or image segmentation, such as one including a convolutional neural network or other types of computer vison networks for deep learning. The artificial intelligence model may be provided with a dataset of segmented two-dimensional ultrasound images.

A machine learning model (e.g., neural network (NN) such as a convolutional neural network, a You Only Look Once (YOLO) deep neural network or a Transformer based neural network architecture) can be trained by using a training dataset. Herein, the training can mean a process of determining weights and parameters of the neural network in order to achieve the object detection from the image input data. As a representative example of the parameter of the NN, there can be a weight given to a synapse or a bias applied to a neuron. Changing hyper-parameters and the neural network architecture can also be done during the training (convolutional neural network architecture can have multiple layers and kernels of various sizes, number of key points within a YOLO model can be varied, etc.) in order to optimize the object detection result.

Examples of unsupervised learning (a subcategory of machine learning models) include but are not limited to clustering and independent component analysis.

Examples of artificial neural networks using unsupervised learning include, but are not limited to, a generative adversarial network (GAN) and an autoencoder (AE), a convolutional neural network (CNN), or a Transformer based neural network, etc.

Examples of supervised learning (a subcategory of machine learning models) include Supervised Vector Machine and Regression Tree.

Examples of artificial neural networks using supervised learning include convolutional neural networks (CNNs), recurrent neural networks (RNNs), long short-term memory (LSTM) and Transformer based neural network and Vision Transformer neural network.

An exemplary deep neural network is a convolutional neural network.

The three-dimensional (3D) reconstruction module 540 includes program code stored in memory 102 that, when executed by the processor 101, causes the processor 101 to arrange the segmented two-dimensional ultrasound images based on the determined position values and orientation values of the ultrasound probe 151 corresponding to each of the segmented two-dimensional ultrasound images. The position values and orientation values can be mapped to a corresponding relative position and relative orientation of each of the segmented two-dimensional ultrasound images with respect to one another. As such, by arranging and orienting each of the segmented two-dimensional ultrasound images with respect to one another, the 3D reconstruction module 540 causes the processor 101 to reconstruct a three-dimensional model of the atherosclerotic plaque, having been identified in the segmented two-dimensional ultrasound images, optionally within a 3D model of the vasculature that may equally be reconstructed, the segmented two-dimensional ultrasound images leading to an estimation of the shape and volume of the 3D object of the atherosclerotic plaque and, optionally, the vasculature.

In some embodiments, the 3D reconstruction module 540 further causes the processor 101 to identify portions of the 3D reconstruction of the atherosclerotic plaque and, optionally the vasculature, that requires more data to define with sufficient accuracy the shape and volume of the 3D object(s) and/or the orientation and position of the ultrasound probe with respect to the different 2D ultrasound images. The 3D reconstruction module 540 may generate a prompt to a user (e.g. a visual queue, a sound, etc.) to request that the user generate more data using the ultrasound probe 151, resulting in further two-dimensional ultrasound images that may be segmented, analyzed, and arranged with respect to the previously acquired and segmented two-dimensional ultrasound images. A threshold value of confidence or accuracy with respect to the structure of the 3D reconstruction of the atherosclerotic plaque and, optionally the vasculature, may be defined, where attaining this threshold value of confidence or accuracy causes the 3D reconstruction module 540 to no longer cause the processor 101 to issue commands for generating prompts for the user to acquire more ultrasound data. In some instances, a signal (e.g. a message) may be generated for the user, indicative that sufficient ultrasound data has been acquired for reconstructing the 3D reconstruction of the atherosclerotic plaque and, optionally the vasculature.

In some instances, the 3D reconstruction module 540 may further cause the processor to receive one or more two-dimensional ultrasound images of a length of the vasculature to be further analyzed for purposes of reconstructing the three-dimensional reconstruction of the atherosclerotic plaque and, optionally the vasculature.

The program code of the 3D reconstruction module 540 may further cause the processor 101 to determine certain properties of the reconstructed three-dimensional atherosclerotic plaque from the three-dimensional atherosclerotic plaque object. Such properties may include, but are not limited to, volume of the atherosclerotic plaque, shape of the atherosclerotic plaque, stenosis caused by the atherosclerotic plaque and echogenicity of the atherosclerotic plaque.

The 3D reconstruction module 540 may cause the processor 101 to display the 3D reconstruction of the atherosclerotic plaque and, optionally of the vasculature, on a display 104 of the computing device 100 (for viewing by a user).

The risk evaluation module 550 includes program code stored in memory 102 that, when executed by the processor 101, causes the processor 101 to establish a risk of a thromboembolic event for a subject. The risk evaluation module 550 causes the processor 101 to calculate the risk from, but is not limited to, one or more of the following:
the subject's clinical or patient information, such as weight, age, gender, biological sex, body mass index, ethnicity, history of smoking, history of cardiovascular events, cholesterol level, glucose level, blood pressure, etc.
the subject's medical history, such as a number of hospitalizations, a length of stay of hospitalization, a presence of a transient ischemic attack event, a presence of an amaurosis fugax event, a presence of a stroke event, etc.;
characteristics of the three-dimensional reconstruction of the atherosclerotic plaque such as volume and shape, stenosis caused by the atherosclerotic plaque, plaque composition, echogenicity of the atherosclerotic plaque, blood flow dynamics and characteristics, etc.;
a number of reconstructed three-dimensional atherosclerotic plaques in vasculature;
density of plaques in vasculature and location of the atherosclerotic plaques in vasculature.

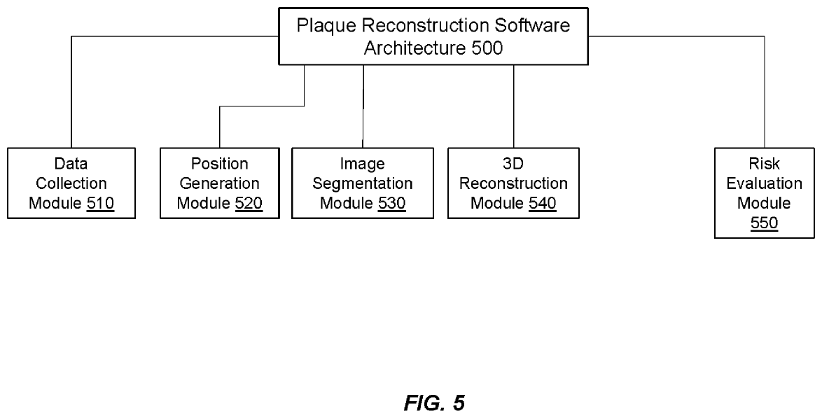

Reference is now made to FIG. 6, illustrating an exemplary method 600 of generating a three-dimensional reconstruction of an atherosclerotic plaque from a plurality of two-dimensional ultrasound images of vasculature. The method 600 may be performed by device 100, or any other device in accordance with the present teachings.

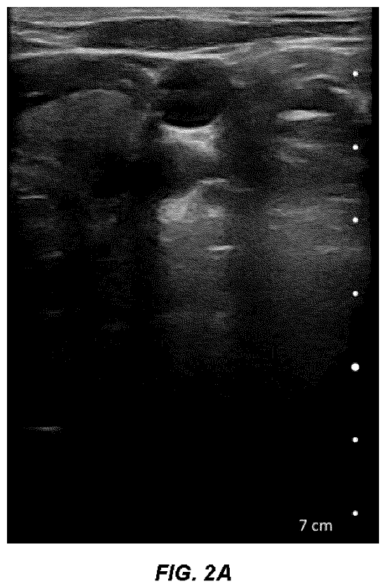

A plurality of two-dimensional ultrasound images of vasculature is received at step 610. An example of a two-dimensional ultrasound image of vasculature, showing a cross-section of vasculature, is found at FIG. 2. The vasculature shown in FIG. 2 is the common carotid artery. However, it will be understood that the vasculature captured using ultrasound may be of any other vasculature without departing from the present teachings.

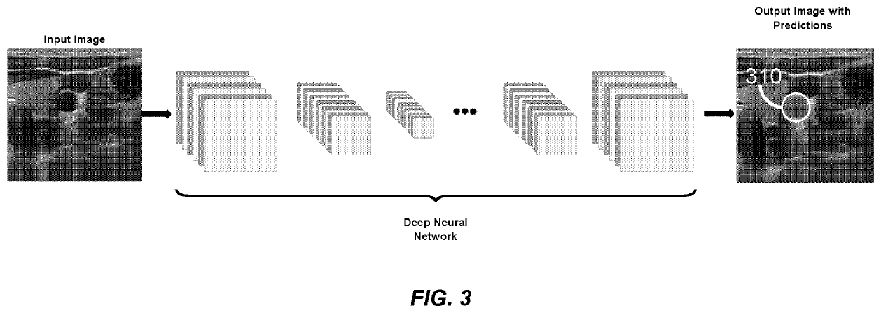

The two-dimensional ultrasound images are generated from ultrasound data originating from an ultrasound probe 151. The ultrasound probe 151 is positioned on skin of a subject at the location of the vasculature of interest, as illustrated in FIG. 4. The ultrasound probe 151 may be connected to an ultrasound machine as is known in the art to visualize an ultrasound image from the data produced by the ultrasound probe 151. It will be understood that the plurality of two-dimensional ultrasound images may form a, or result from, video or image stream, composed of a sequential stream of the two-dimensional ultrasound images.

An entire length of the vasculature may be scanned to capture possible locations for atherosclerotic plaque formation.

The ultrasound probe 151 is moved along the skin of the subject, where orientation and position of the ultrasound probe varies to generate resulting two-dimensional ultrasound images of the vasculature, showing cross-sections taken at different positions and orientations of the vasculature, as shown in FIG. 4.

The two-dimensional ultrasound images may be B-mode ultrasound images. Other exemplary two-dimensional ultrasound images may be M-mode, Doppler, Pulse Wave, shear wave elastography, etc. In some examples, the two-dimensional ultrasound images may be in a DICOM format.

Positional data of the ultrasound probe 151 generating the ultrasound data resulting in the two-dimensional ultrasound images is received at step 620. The positional data is generated by one or more position sensors 152 providing information on a position and/or change in position of the ultrasound probe 151. In some instances, the position sensors 152 may be joined to the ultrasound probe 151. For instance, the one or more position sensors 152 may be joined or joinable to the ultrasound probe 151 using a mounting, the mounting connecting the one or more position sensors to the ultrasound probe. In some instances, the one or more position sensors 152 may be joined to the ultrasound probe 151 by being directly integrated into the ultrasound probe.

The one or more position sensors 152 may be, but are not limited to, one or more of:
one or more RGB cameras (e.g. functioning as optical trackers) generating an image stream as the positional data;
one or more infrared cameras generating an infrared image stream as the positional data;
one or more gyroscopes generating rotational motion information as the positional data;
one or more electromagnetic sensors generating electromagnetic readings as the positional data.

The positional data may include, or may be related to, (e.g. in metadata) time information or a timestamp, such that the positional data can be associated with its corresponding two-dimensional ultrasound image (where the positional data may be matched with its corresponding two-dimensional ultrasound image using a respective timestamp of the positional data and the respective timestamp of the two-dimensional ultrasound image). In some instances, other identifying information (e.g. a string of characters forming an identifier) may be used to match the positional data with its respective two-dimensional ultrasound image. In some instances, the time information may be generated at the computing device receiving the data from the ultrasound probe and the position sensors (e.g. by adding a timestamp corresponding to a time of receipt of the positional data and of the two-dimensional ultrasound images). In some embodiments, the computing device 100 may match the positional data with corresponding two-dimensional ultrasound images, when the positional data and the two-dimensional ultrasound images are received from the position sensor(s) 152 and the ultrasound probe 151, respectively, in real time.

The received plurality of two-dimensional ultrasound images are then segmented at step 630 to identify the atherosclerotic plaques appearing in the two-dimensional ultrasound images, separating the atherosclerotic plaques from the surrounding vasculature tissue. The surrounding vasculature tissue may be further defined in the two-dimensional ultrasound images.

In some instances, a boundary line may be traced around the identified atherosclerotic plaque, and optionally, around the surrounding vasculature tissue.

In some instances, a trained artificial intelligence model may perform the segmentation of the two-dimensional ultrasound images to identify the atherosclerotic plaque(s), and optionally, around the surrounding vasculature tissue. The artificial intelligence model may be trained using a dataset of ultrasound images that have been previously segmented to identify captured atherosclerotic plaque(s), and/or surrounding vasculature tissue.

In some examples, the segmentation of the two-dimensional ultrasound images may be performed using a software algorithm configured for object recognition to identify patterns or identifiers for locating the atherosclerotic plaque(s), and optionally, around the surrounding vasculature tissue.

Position and orientation values of the ultrasound probe 151 may be generated at step 640 from the positional data. In some embodiments, the robust features captured by the position sensors 152 (the robust features identified in the image stream generated by a position sensor 152 that is a camera) are tracked over time and the position and orientation of the ultrasound probe is triangulated resultingly over time. In an example using visual tracking, such as with panoramic image stitching, a camera is able to locate and triangulate visual features in its environment, thereby enabling it to determine its own position and orientation in relation to these visual features. In the case of magnetic tracking, an off-board transmitter (e.g., 152B) sends out pulses that are reflected by the trackers affixed to the ultrasound probe 151. In some embodiments, a use of three or more position sensors 152 (e.g. trackers) allows for localization of the probe 151 (e.g. through a tracker) in 3D space (similarly to locating a cell phone using a network of towers).

For instance, when the positional data includes an image stream generated by a camera joined to the ultrasound probe 151, a change in position, or of size, of references points or objects visible in virtual or image space can be correlated to a change in position and orientation of the ultrasound probe. Further positional data, such as acceleration readings generated by an accelerometer, can further provide indication regarding a change in position of the ultrasound probe.

For instance, an exemplary method of generating the position and orientation information is described in A. Benjamin, 3D organ property mapping using freehand ultrasound scans, 2020, incorporated herein by reference (accessible via https://scholar.google.com/citations?view_op=view_citation&hl=en&user=6LTRb8wA AAAJ&citation_for_view=6LTRb8wAAAAJ:MXK_kJrjxJIC, last accessed on Feb. 28, 2023). As described, in one example, a fusion of multiple low-cost sensors in a Bayesian framework localizes a conventional ultrasound probe with respect to the room or the subject's body; localizing the probe relative to the body of the subject may be achieved by using the subject's superficial vasculature as a natural encoding system. Segmented 2D ultrasound images and quantitative 2D speed of sound maps obtained using numeric inversion may be stitched together to create 3D property maps.

With the determined position values and orientation values of the ultrasound probe 151, a corresponding position and orientation of a two-dimensional ultrasound image may be generated, where the position and orientation of the two-dimensional ultrasound image is dependent on the view found in the two-dimensional ultrasound image that is generated from the ultrasound probe 151, where the view captured in the two-dimensional ultrasound probe 151 is dependent on the properties of the ultrasound probe 151. A transformation of the position and orientation of the ultrasound probe 151 to a position and orientation of the two-dimensional ultrasound images may be achieved through use of a function yielding a field of view of the two-dimensional ultrasound image based on the orientation and position of the ultrasound probe 151.

The positions and orientations of the ultrasound probe 151 (or if the generated two-dimensional images), if not already matched, may be matched to each of the plurality of segmented two-dimensional ultrasound images.

A three-dimensional representation of the atherosclerotic plaque may then be generated at step 650. Each 2D ultrasound image has a corresponding position and orientation. As such, one or more of the pixel within the image is converted to a point in 3D space. The transformation from 2D space to 3D space is represented by two linear transformations: one from the ultrasound probe 151 to the one or more position sensors 152 joined to the ultrasound probe 151, and the other from the one or more position sensors 152 to the ambient world.

The arrangement of the two-dimensional segmented ultrasound images in the 3D map, representing cross-sections of the vasculature, based on position and orientation, results in an arrangement of cross-sections of the vasculature, with the segmented atherosclerotic plaque, visible on the two-dimensional ultrasound images, that provides information on the 3D structure of the atherosclerotic plaque through the sum of the positioned and oriented two-dimensional segmented ultrasound images. A volume is then generated for the segmented atherosclerotic plaque from the sum of the positioned and oriented two-dimensional segmented ultrasound images, defined by the identified atherosclerotic plaque appearing in each of the two-dimensional segmented ultrasound images.

In some embodiments, one or more two-dimensional ultrasound images of a longitudinal view of the vasculature may be received and processed for reconstructing the three-dimensional model of the atherosclerotic plaque.

In some embodiments, a visual indicator may be presented to the user (e.g. on a display), for requesting that the user continue to generate more ultrasound data using the ultrasound probe 151, to further improve the accuracy of the reconstruction of the three-dimensional model of the atherosclerotic plaque. The user may be further prompted to acquire the ultrasound data until a confidence threshold for the properties of the three-dimensional model of the atherosclerotic plaque is achieved. The underlying concept is that the uncertainty in the estimated position and orientation of the 2D ultrasound images is propagated to the underlying 3D reconstruction; depending upon the level of uncertainty, the user may be prompted to perform additional scans in specific regions. When a threshold level of certainty has been met, the user may no longer be prompted to acquire additional 2D ultrasound frames by scanning the subject.

In some instances, for vasculature, a length of the vasculature to be scanned and generated into a 3D reconstruction may be defined. For vasculature, due to its significant length, an entirety of the vasculature may not have to be scanned. Instead, specific portions, of a designated length, of the vasculature may be scanned (e.g. a portion of the carotid artery to identify possible plaques). In these embodiments, a first input may be entered by the user indicating the length of the vasculature to be scanned. This may be defined through an initial pass identifying the length of vasculature to be scanned, where the start and end point of the first pass may define the length of the vasculature to be analyzed for purposes of the 3D reconstruction of the vasculature.

When feedback is given to the user to perform further scans of the vasculature to reduce the level of uncertainty regarding position and orientation (or to improve the resolution of the 3D reconstruction of the vasculature), the user may be prompted to further scan the vasculature falling within the length of the vasculature that has been previously defined.

In some instances, the three-dimensional model of the atherosclerotic plaque may be further analyzed to determine a volume of the corresponding atherosclerotic plaque.

In some instances, the three-dimensional model of the atherosclerotic plaque may be further analyzed to determine a shape of the corresponding atherosclerotic plaque.

In some instances, the three-dimensional model of the atherosclerotic plaque may be further analyzed to determine a thickness of the corresponding atherosclerotic plaque.

In some instances, the three-dimensional model of the atherosclerotic plaque may be further analyzed to determine a volume of the corresponding atherosclerotic plaque.

In some instances, the three-dimensional model of the atherosclerotic plaque may be further analyzed to determine a stenosis or blockage caused by the corresponding atherosclerotic plaque.

In some instances, the three-dimensional model of the atherosclerotic plaque may be further analyzed to determine the pixel intensity heterogeneity of the corresponding atherosclerotic plaque.

Like step 650, a three-dimensional reconstruction of the surrounding vasculature, visible in the two-dimensional ultrasound images, containing the atherosclerotic plaque, may be performed at step 660. Like step 650, the three-dimensional reconstruction of the surrounding vasculature is performed from segmented two-dimensional ultrasound images identifying the surrounding vasculature tissue. In some instances, the three-dimensional reconstruction of the surrounding vasculature may contain the three-dimensional reconstruction of the atherosclerotic plaque.

Further information may be determined from the three-dimensional reconstruction of the surrounding vasculature, such as, for instance, a diameter of the vasculature, and/or an intimal-medial thickness of the vasculature.

The three-dimensional reconstruction of the atherosclerotic plaque may be used to determine a risk of a thromboembolic event at step 670. A risk score of a thromboembolic event may be generated, but is not limited to, from one or more of:
the subject's clinical or patient information, such as weight, age, gender, biological sex, ethnicity, body mass index, history of smoking, history of cardiovascular events, cholesterol level, glucose level, blood pressure, etc.;
the subject's medical history, such as a number of hospitalizations, a length of stay of hospitalization, a presence of a transient ischemic attack event, a presence of an amaurosis fugax event, and a presence of a stroke event;
characteristics of the three-dimensional reconstruction of the atherosclerotic plaque such as volume and shape, stenosis caused by the atherosclerotic plaque and echogenicity of the atherosclerotic plaque, plaque composition, blood flow dynamics and characteristics, etc.;
number of reconstructed three-dimensional atherosclerotic plaques in vasculature;
pixel density of plaques in vasculature and location of the atherosclerotic plaques in vasculature.

As such, method 600 permits obtaining three-dimensional information of an atherosclerotic plaque appearing in vasculature of a subject, indicative of a risk of a thromboembolic event, achieved by using equipment readily available in a healthcare setting (i.e. a two-dimensional (B Mode) ultrasound machine). The generating of the three-dimensional information on the atherosclerotic plaque is possible as a result of determining a position and orientation of the ultrasound probe that produced the ultrasound data that resulted in the two-dimensional ultrasound images capturing cross-sections of the atherosclerotic plaque.

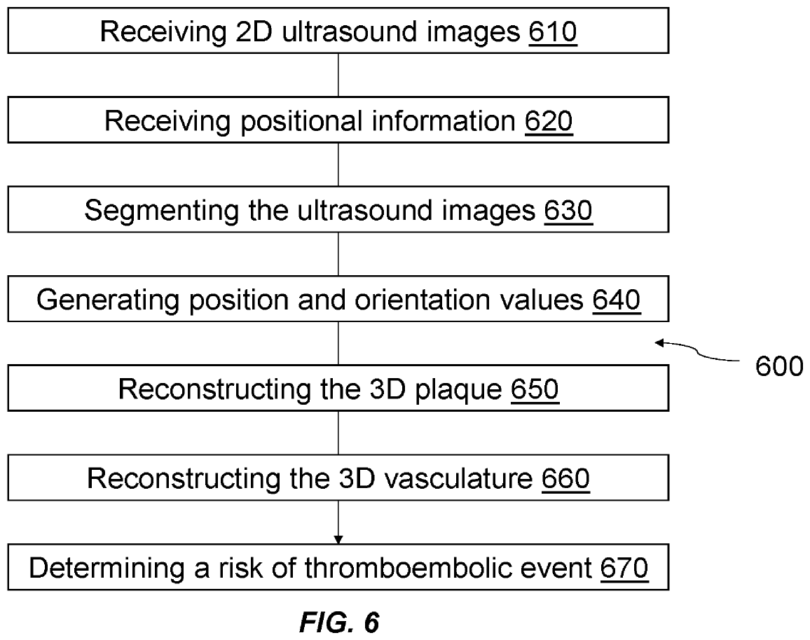

Reference is now made to FIG. 7, illustrating an exemplary method 700 of computing a three-dimensional representation of an atherosclerotic plaque from a plurality of two-dimensional ultrasound images of vasculature. The method 700 may be performed by device 100, or any other device in accordance with the present teachings.

In some instances, calibrating 702 the system may be necessary. The calibrating 702 is meant to ensure that the ultrasound probe 151 and the position sensors 152 are calibrated with one another (e.g., position and movements of the ultrasound probe 151 are properly captured by the position sensors 152). The calibrating 702 may involve performing a calibration sequence comprising various measurements and positioning the ultrasound probe 151. For instance, the calibration sequence may be provided to the operator through the GUI 2150.

Two-dimensional ultrasound images of vasculature are acquired 710 from the ultrasound probe 151. Each image is associated to positional data of the ultrasound probe 151 relative to the vasculature at the time of image acquisition. An example of a two-dimensional ultrasound image of vasculature, showing a cross-section of vasculature, is found at FIG. 2. The vasculature shown in FIG. 2 is the common carotid artery. However, it will be understood that the vasculature captured using ultrasound may be of any other vasculature without departing from the present teachings.

The two-dimensional ultrasound images are acquired from ultrasound data originating from an ultrasound probe 151. The ultrasound probe 151 is positioned on skin of a subject at the location of the vasculature of interest, as illustrated in FIG. 4. The ultrasound probe 151 may be connected to an ultrasound machine as is known in the art to visualize an ultrasound image from the data produced by the ultrasound probe 151. It will be understood that the plurality of two-dimensional ultrasound images may form a, or result from, video or image stream, composed of a sequential stream of the two-dimensional ultrasound images.

An entire length of the vasculature may be scanned to capture possible locations for atherosclerotic plaque formation.

The ultrasound probe 151 is moved along the skin of the subject, where orientation and position of the ultrasound probe 151 varies to generate resulting two-dimensional ultrasound images of the vasculature, showing cross-sections taken at different positions and orientations of the vasculature, as shown in FIG. 4.

The two-dimensional ultrasound images may be B-mode ultrasound images. Other exemplary two-dimensional ultrasound images may be M-mode, Doppler, Pulse Wave, shear wave elastography, etc. In some examples, the two-dimensional ultrasound images may be in a DICOM format.

The positional data associated to the two-dimensional ultrasound images of the ultrasound probe 151 may be generated 720 in various ways by one or more position sensors 152. The manner in which the positional data is generated 720 does not affect the remainder of the features described herein. Some techniques shown herein may be synergetic, which does not create a requirement for such exemplary techniques. As previously mentioned, in some instances, the position sensors 152 may be joined to the ultrasound probe 151. For instance, the one or more position sensors 152 may be joined or joinable to the ultrasound probe 151 using a mounting connecting the one or more position sensors to the ultrasound probe 151. In some instances, the one or more position sensors 152 may be joined to the ultrasound probe 151 by being directly integrated into the ultrasound probe. the position sensors 152 may also be distributed between a component mounted on or joined to the ultrasound probe 151 and a component mounted on or joined to equipment of the environment that is kept stationary during the acquiring 710 of the 2D ultrasound images.

As previously mentioned, the one or more position sensors 152 may be, but are not limited to, one or more of:
one or more RGB cameras (e.g. functioning as optical trackers) generating an image stream as the positional data;
one or more infrared cameras generating an infrared image stream as the positional data;
one or more gyroscopes generating rotational motion information as the positional data; and
one or more electromagnetic sensors generating electromagnetic readings as the positional data. For instance, the position sensors 152 comprise an emitter and a receiver distributed between a fixed point and a mobile point that follows the ultrasound probe 151 movements (i.e., with the emitter and receiver being respectively either fixed or mobile).

The positional data may include, or may be related to, (e.g. in metadata) time information or a timestamp, such that the positional data can be associated with its corresponding two-dimensional ultrasound image (where the positional data may be matched with its corresponding two-dimensional ultrasound image using a respective timestamp of the positional data and the respective timestamp of the two-dimensional ultrasound image). In some instances, other identifying information (e.g. a string of characters forming an identifier) may be used to match the positional data with its respective two-dimensional ultrasound image. In some instances, the time information may be generated at the device 100 receiving the data from the ultrasound probe 151 and the position sensors 152 (e.g. by adding a timestamp corresponding to a time of receipt of the positional data and of the two-dimensional ultrasound images). In some embodiments, the device 100 may match the positional data with corresponding two-dimensional ultrasound images, when the positional data and the two-dimensional ultrasound images are received from the position sensor(s) 152 and the ultrasound probe 151, respectively, in real time.

The acquired 710 plurality of two-dimensional ultrasound images are segmented 730 considering varying acoustic signatures between one or more plaque atherosclerotic bodies and vasculature tissue. In some instances, a boundary line may be traced around the identified atherosclerotic plaque, and optionally, around the surrounding vasculature tissue.

In some instances, a trained artificial intelligence model may perform the segmentation of the two-dimensional ultrasound images to identify the atherosclerotic plaque(s), and optionally, around the surrounding vasculature tissue. The artificial intelligence model may be trained using a dataset of ultrasound images that have been previously segmented to identify captured atherosclerotic plaque(s), and/or surrounding vasculature tissue.

In some examples, segmenting 730 the two-dimensional ultrasound images may be performed using a software algorithm configured for object recognition to identify patterns or identifiers for locating the atherosclerotic plaque(s), and optionally, around the surrounding vasculature tissue.

Location and orientation values over time are computed 740 from the positional data. In some embodiments, the robust features captured by the position sensors 152 (the robust features identified in the image stream generated by a position sensor 152 that is a camera) are tracked over time and the location and orientation of the ultrasound probe is triangulated resultingly over time. In an example using visual tracking, such as with panoramic image stitching, a camera is able to locate and triangulate visual features in its environment, thereby enabling it to determine its own position and orientation in relation to these visual features. In the case of magnetic tracking, an off-board transmitter (e.g., 152B) sends out pulses that are reflected by the trackers affixed to the ultrasound probe 151. In some embodiments, a use of three or more position sensors 152 (e.g. trackers) allows for localization of the probe 151 (e.g. through a tracker) in 3D space (similarly to locating a cell phone using a network of towers).

For instance, when the positional data includes an image stream generated by a camera joined to the ultrasound probe 151, a change in position, or of size, of references points or objects visible in virtual or image space can be correlated to a change in position and orientation of the ultrasound probe. Further positional data, such as acceleration readings generated by an accelerometer, can further provide indication regarding a change in position of the ultrasound probe.

For instance, an exemplary method of generating the position and orientation information is described in A. Benjamin, 3D organ property mapping using freehand ultrasound scans, 2020, incorporated herein by reference (accessible via https://scholar.google.com/citations?view_op=view_citation&hl=en&user=6LTRb8wA AAAJ&citation_for_view=6LTRb8wAAAAJ:MXK_kJrjxJIC, last accessed on Feb. 28, 2023). As described, in one example, a fusion of multiple low-cost sensors in a Bayesian framework localizes a conventional ultrasound probe with respect to the room or the subject's body; localizing the probe relative to the body of the subject may be achieved by using the subject's superficial vasculature as a natural encoding system. Segmented 2D ultrasound images and quantitative 2D speed of sound maps obtained using numeric inversion may be stitched together to create 3D property maps.

Skilled persons will readily understand the computed 740 location values and orientation values over time are important in the context of a subsequent computing 750 of plaque, detailed hereinbelow. Depending on expected data for the computing 750, the computing 740 may therefore involve computing a location of the ultrasound probe 151 and an orientation of the ultrasound probe 151 from or the computing 740 may involve computing location values and orientation values of the 2D images without computing or conserve location and orientation values of the ultrasound probe 151. In some other instances, both location and orientation values of the ultrasound probe 151 and location and orientation values of the 2D ultrasound images may be performed, e.g., when the computing 750 is so configured.

From the computed 740 location values and orientation values and the segmented 730 images, a three-dimensional representation of the atherosclerotic plaque in at least one blood vessel in the vasculature is computed 750. Various techniques may be used during the computing 750. For instance, one or more of the pixels within the image may be converted to a point in 3D space (e.g., FIG. 2B). In some instances, the computing 750 is performed for the vasculature at the same time as the at least one blood vessel and a reconstruction of the three-dimensional representation of the atherosclerotic plaque in the vasculature is therefore obtained. In some instances, a three-dimensional reconstruction of the surrounding vasculature, visible in the two-dimensional ultrasound images, containing the atherosclerotic plaque, may be computed 760 separately. Computing 760 of the three-dimensional reconstruction of the vasculature may performed from the segmented 730 two-dimensional ultrasound images. In some instances, the three-dimensional reconstruction of the surrounding vasculature may contain the three-dimensional representation of the atherosclerotic plaque.

In some instances, the two-dimensional segmented ultrasound images may be mapped in 3D space, representing cross-sections of the vasculature, based on location and orientation values. In some implementations. It may be helpful to visibly represent the two-dimensional ultrasound images that form the basis from the computed 750 3D representation of the atherosclerotic plaque. In some implementations, the computed 750 3D representation of the atherosclerotic plaque may be made visible.

In some embodiments, one or more two-dimensional ultrasound images of a longitudinal view of the vasculature may be received and processed for reconstructing the three-dimensional representation of the atherosclerotic plaque.

In some embodiments, a visual indicator may be presented to the operator (e.g. on a display through the GUI module 2150), for requesting that the operator continue to generate more ultrasound data using the ultrasound probe 151, to further improve the accuracy of the representation of the three-dimensional atherosclerotic plaque. The operator may be further prompted to acquire the ultrasound data until a confidence threshold for the properties of the three-dimensional representation of the atherosclerotic plaque is achieved. The underlying concept is that the uncertainty in the estimated position and orientation of the 2D ultrasound images is propagated to the underlying 3D representation. Depending upon the level of uncertainty, the operator may be prompted to perform additional scans in specific regions. When a threshold level of certainty has been met, the operator may no longer be prompted to acquire additional 2D ultrasound frames by scanning the subject.

In some instances, for vasculature, a length of the vasculature to be scanned and generated into a 3D reconstruction may be defined. For vasculature, due to its significant length, an entirety of the vasculature may not have to be scanned. Instead, specific portions, of a designated length, of the vasculature may be scanned (e.g. a portion of the carotid artery to identify possible plaques). In these embodiments, a first input may be entered by the operator indicating the length of the vasculature to be scanned. The first input may be defined through an initial pass identifying the length of vasculature to be scanned, where the start and end point of the first pass may define the length of the vasculature to be analyzed for purposes of the 3D reconstruction of the vasculature.

When feedback is given to the operator to perform further scans of the vasculature to reduce the level of uncertainty regarding position and orientation (or to improve the resolution of the 3D reconstruction of the vasculature), the operator may be prompted to further scan the vasculature falling within the length of the vasculature that has been previously defined.

In some instances, the three-dimensional representation of the atherosclerotic plaque may be further analyzed to determine a volume of the corresponding atherosclerotic plaque.

In some instances, the three-dimensional representation of the atherosclerotic plaque may be further analyzed to determine a shape of the corresponding atherosclerotic plaque.

In some instances, the three-dimensional representation of the atherosclerotic plaque may be further analyzed to determine a thickness of the corresponding atherosclerotic plaque.

In some instances, the three-dimensional representation of the atherosclerotic plaque may be further analyzed to determine a volume of the corresponding atherosclerotic plaque.

In some instances, the three-dimensional representation of the atherosclerotic plaque may be further analyzed to determine a stenosis or blockage caused by the corresponding atherosclerotic plaque.

In some instances, the three-dimensional representation of the atherosclerotic plaque may be further analyzed to determine the pixel intensity heterogeneity of the corresponding atherosclerotic plaque.

Further information may be determined from the three-dimensional reconstruction of the surrounding vasculature, such as, for instance, a diameter of the vasculature, and/or an intimal-medial thickness of the vasculature.

The three-dimensional representation of the atherosclerotic plaque may be used to compute 770 a risk score of a thromboembolic event from one or more of:
the subject's clinical or patient information, such as weight, age, gender, biological sex, ethnicity, body mass index, history of smoking, history of cardiovascular events, cholesterol level, glucose level, blood pressure, etc.;
the subject's medical history, such as a number of hospitalizations, a length of stay of hospitalization, a presence of a transient ischemic attack event, a presence of an amaurosis fugax event, and a presence of a stroke event;
characteristics of the three-dimensional reconstruction of the atherosclerotic plaque such as volume and shape, stenosis caused by the atherosclerotic plaque and echogenicity of the atherosclerotic plaque, plaque composition, blood flow dynamics and characteristics, etc.;
number of reconstructed three-dimensional atherosclerotic plaques in vasculature;
pixel density of plaques in vasculature and location of the atherosclerotic plaques in vasculature.

As such, the method 700 permits characterization through three-dimensional information of an atherosclerotic plaque appearing in a vasculature of a subject. The method 700 may further provide indication of a risk of a thromboembolic event by using equipment readily available in a healthcare setting (i.e., a two-dimensional (B Mode) ultrasound machine).

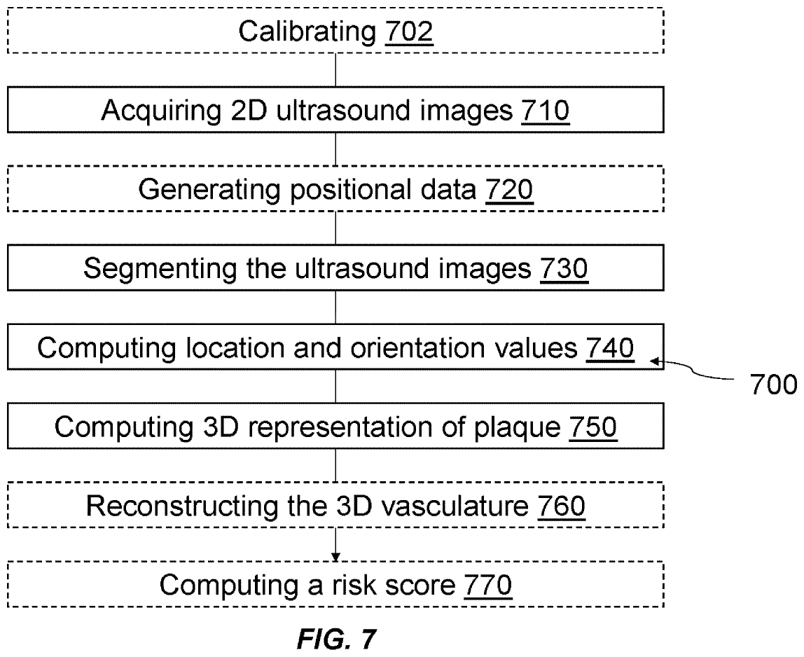

A medical imaging system used in the context of the example of the method 800 of FIG. 8 may be provided.

The system comprises a position-tracking sensor designed to be affixed to an ultrasound probe. The sensor is configured to record precise positional data during the ultrasound scanning process.

The system also comprises an ultrasound probe that, when equipped with the aforementioned sensor, is positioned 810 at a predetermined starting point on the transverse plane of a target artery, such as the carotid artery.

Imaging data is acquired 820 from the ultrasound probe and spatial positioning data may be further generated 840 from the sensor.

An optional step 860 may be provided allowing the operator to conduct additional sweeps. The initial sweep and, in some instances, the additional sweeps may then be used to compile 870 an enhanced dataset of imaging and positional data, thereby enabling accurate three-dimensional reconstructions of the artery.

Computing 880 of a three-dimensional representation of the atherosclerotic plaque in the vasculature may then be performed from the set of two-dimensional ultrasound images and positional data. The computing 880 may be performed by a processing unit, which may be a cloud-based platform or an integrated software suite, that receives the combined imaging and positional data. The processing unit may be responsible for processing the received data to construct a dynamic three-dimensional model of the artery. The model includes detailed representations of the arterial walls, any present plaque, or other pathologies.

An analysis algorithm, e.g., embedded within the processing unit, may examine the generated 3D representation to provide quantitative measurements. The quantitative measurements may include, without being limited thereto, the dimensions of the vessel walls and plaque characteristics, ultimately culminating in a comprehensive analysis report for clinical evaluation.

Quick and efficient vascular scans may therefore be enabled, reducing examination time by at least 4-fold significantly compared to conventional methods, and providing enhanced data for the creation of highly accurate three-dimensional vascular models for diagnostic and monitoring purposes.

During a traditional ultrasound examination related to arterial plaque, the operator, holding the ultrasound transducer, applies gentle pressure and moves it over the skin surface to find the best angle for visualizing the artery and the arterial walls.

Live ultrasound images are observed on a monitor. The operator adjusts the position, angle, and settings of the transducer to optimize the image quality, focusing on areas where plaques are suspected.

The operator employs various ultrasound modes, such as Duplex ultrasound, which combines B-mode for structural visualization and Color Doppler to assess the speed and direction of blood flow within vessels. Spectral and Power Doppler ultrasound can be used for more detailed information on blood flow velocity, to thoroughly examine the artery and identify plaques.

Specific segments of the artery are scanned systematically to ensure comprehensive coverage and to document the presence, location, size, and characteristics of any plaques. The images are then saved.

The saved images and data are analyzed to determine the presence, size, composition, and exact location of the plaque within the artery. The echogenicity (brightness) and texture of the plaque on the ultrasound images can provide information about its composition. Measurements of the plaque and the arterial lumen, along with blood flow velocity measurements, are taken to assess the degree of stenosis (narrowing of the artery).

As such, a traditional examination procedure requires an operator to be knowledgeable enough in arterial plaque to build proper document thereof and experience indicates that it takes at least 20 minutes.

By comparison, with particular reference to the method 800, but also generally considering the present disclosure, in 10 minutes or less, all images required to ensure a reliable 3D representation of plaque in a blood vessel may be acquired using ultrasound equipment having similar imaging capabilities compared with traditional methods.

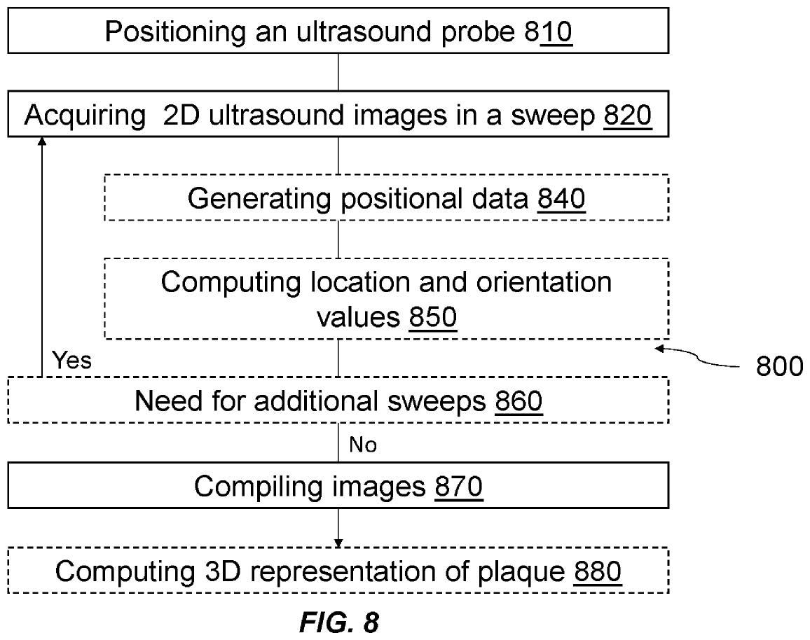

With particular reference to FIG. 9, a guidance and feedback mechanism may be provided to assist operators during medical imaging procedures, thereby enhancing the accuracy and quality of the scans produced.

The guidance and feedback mechanism may be configured to provide both real-time and retrospective feedback to the operator during and post-acquisition, respectively. Real-time feedback may be presented through a user interface that delivers multimodal cues, which may be auditory, visual, or a combination thereof. These cues are generated in response to the analysis of scanning parameters, offering guidance for the localization of anatomical structures such as arteries, and for the adjustment of imaging settings including, but not limited to, ultrasound image gain, contrast, or brightness.

A method 900 may be provided to improve medical scan quality utilizing the guidance and feedback system by identifying 910 one or more parameters affecting the quality of the medical scans. The parameters may comprise: artery location, ultrasound image gain, contrast, or brightness, and image quality metrics. Upon identification 910 one or more parameters, feedback may be provided 920 to the operator through the presentation of intuitive prompts. The prompts may be strategically designed to ensure that the operator can quickly comprehend and execute the necessary adjustments to improve the image quality. An amplitude of the operator feedback may be proportional to an amplitude of a deviation between an expected value of the parameter(s) and an actual value therefor.

Feedback provided 920 to the operator by the system may be specifically tailored to support the operator's decision-making process during scanning thereby increasing the likelihood of informed adjustments being made to scanning parameters in real time, leading to immediate improvements in scan quality. Additionally, the feedback may be provided 920 to the operator as post-acquisition guidance. The system may therefore allow for analysis of scan data after the initial acquisition and offer recommendations for improvements in future scans, contributing to a cycle of continuous quality enhancement.

A method may be provided to simulate and characterize the blood flow in blood vessels. The method may be agnostic to the type and location of the blood vessel.

The method may receive as input a 3D structure or reconstruction of a blood vessel. The 3D structure may be obtained from a variety of sources including but not limited to computed tomography, MRI, or ultrasound. The 3D structure may be input in the form of a point cloud or mesh and can take on a variety of formats including but not limited to STL, OBJ, PLY, IDS.

The method may analyze the 3D structure using a defect elimination algorithm to selectively fill gaps and correct anomalies (e.g., not limited to, holes, tears, and noise). The method may then proceed with enhancing the three-dimensional structure using mesh refinement techniques to provide smooth and accurate representation of a geometry of the blood vessel.

The method may also assign material properties to the 3D structure such as inner vessel wall, outer vessel wall, atherosclerotic plaque or other vessel disease, and surrounding muscles/tissue considering that the fluid in concern is blood with the appropriate, temperature dependent properties (specifically density and viscosity at appropriate body temperatures).

When the input to the algorithm is a point-cloud, the method may further generate a mesh of adequate quality with a spatial resolution necessary to capture macro-fluidics within the blood vessel.

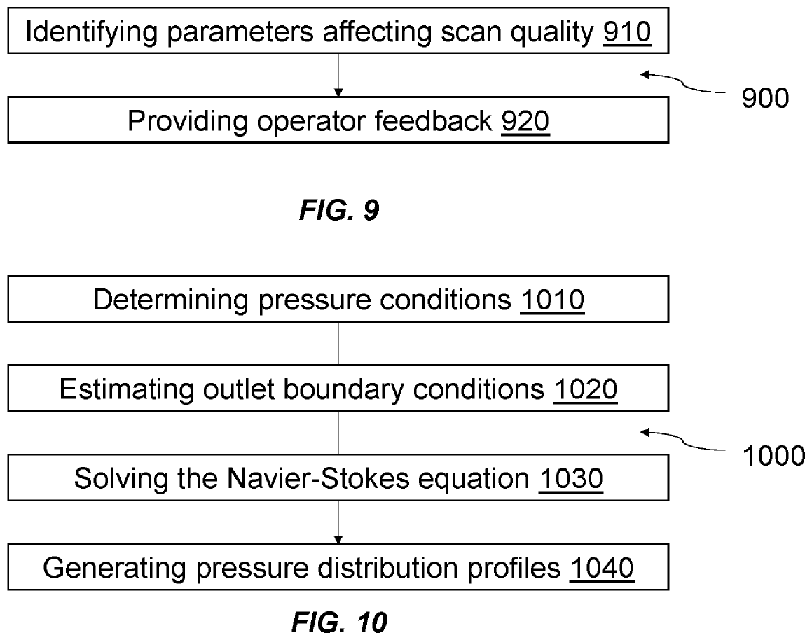

With particular reference to FIG. 10, a method 1000 is provided for setting boundary conditions and solving the Navier-Stokes equation for simulating blood flow in a blood vessel. The method comprises determining 1010 time-varying pressure conditions at an inlet based on an average systolic and diastolic blood pressures of a patient and estimating 1020 outlet boundary conditions employing numerical techniques thereby ensuring consistency with the simulation results. The inlet boundary condition may be a time-varying pressure condition, which may be set based on the average systolic and diastolic blood pressures of the patient. The outlet boundary condition is not set but estimated from the simulation.

The method 1000 may then numerically solve 1030 the Navier-Stokes equation for non-newtonian fluid flow utilizing computational fluid dynamics (CFD) solvers.

The method 1000 may then generate 1070 two field outputs: a pressure distribution field output and a flow velocity profile field output along the length of the vessel.

The method 1000 may solve for the above outputs in the presence or absence of abnormal pathologies (e.g. stenosis), thereby providing the clinician with a complete understanding of flow and pressure within the blood vessel.

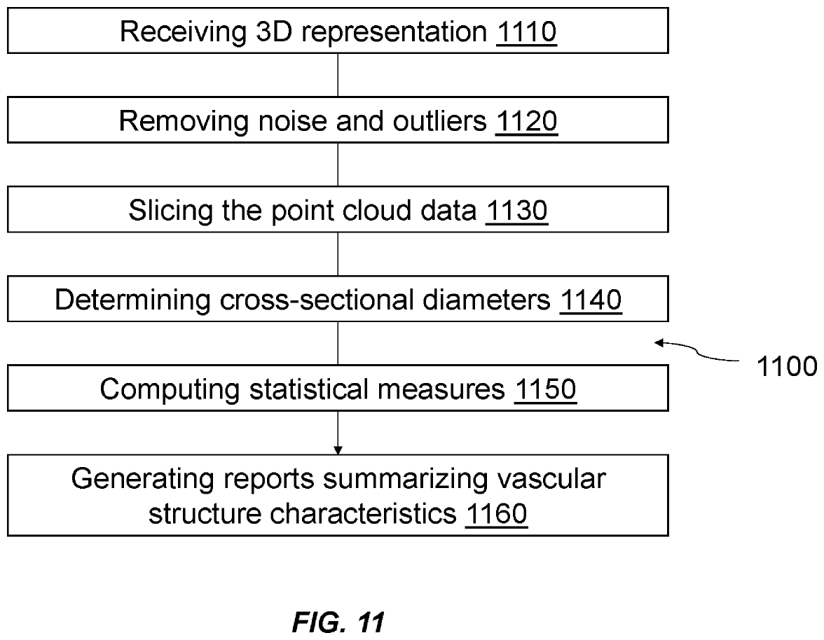

With particular reference to FIG. 11, a method 1100 may be provided to automatically measure diameter and characterization of stenosis and disease from 3D vascular structures obtained from tracked, freehand ultrasound scans. The method 1100 receives 1110 as input a 3D structure or reconstruction of a blood vessel. The 3D structure may be obtained from a variety of sources including but not limited to computed tomography, MRI, or ultrasound.

The method 1100 may accept input in various 3D formats such as mesh, point cloud, or other compatible formats (e.g., STL, PLY, XYZ, OBJ). The method 1100 may remove 1120 noise and outliers from the input point cloud data corresponding to the three-dimensional structure (e.g., not limited to, holes, tears, and noise), e.g., using voxelization, downsampling, or outlier removal. The method 1100 may optionally proceed with enhancing the three-dimensional structure using mesh refinement techniques to provide smooth and accurate representation of a geometry of the blood. Vascular structures from any part of the body can be analyzed, as the method 1100 is location-agnostic. Input point cloud data is preprocessed to remove noise and outliers, ensuring accurate analysis.

Analysis of the underlying vascular structure may be performed automatically to determine the cross-sectional diameter at each point along the vessel's length, there eliminating the need for human intervention. Additionally, statistical insights may be provided such as mean, variance, minimum, maximum, and median diameters. The point cloud is sliced 1130 perpendicular to the vessel's longitudinal axis at regular intervals, creating cross-sectional planes. Each cross-section represents a 2D profile of the vessel at a specific location along its length.

For each cross-section, the diameter is determined 1140, which may be performed by 1) identifying points belonging to the vessel boundary, 2) fitting a shape (e.g., circle, ellipse) to the boundary points using curve fitting or geometric algorithms, and 3) extracting diameter information from the fitted shape, typically by measuring the maximum distance between points or computing the diameter of a circumscribed circle.

Statistical measures such as mean, variance, minimum, maximum, and median diameters are computed 1150 across all cross-sectional diameters. The statistical measures provide insights into the vessel's geometry and diameter variations along its length.

A stenosis measure may then be calculated, indicating blockages, at each point along the vessel's length. The stenosis measure may be derived from geometric properties such as diameter, length, and curvature. The location of maximum stenosis may be identified with associated measurements. Reports summarizing vascular structure characteristics may then be generated 1160.

Additional examples of the method 1100 include computing stenosis for each cross-section based on the reduction in diameter compared to a reference diameter (e.g., healthy vessel diameter). The location of maximum stenosis is identified by pinpointing the cross-section with the most significant reduction in diameter.

Additional measurements such as the extent of stenosis and its distance from reference points (e.g., vessel origin) are computed.

Often, the North American Symptomatic Carotid Endarterectomy Trial and the European Carotid Surgery Trial scoring systems may be employed to calculate stenosis or blockage of the artery.

Stenosis measurements are visualized along the vessel's length, highlighting regions of narrowing or blockage. The reports may be used to summarize stenosis severity, location of maximum stenosis, and associated measurements. Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

1. A method of characterizing an atherosclerotic plaque in vasculature comprising:
acquiring a plurality of two-dimensional ultrasound images of the vasculature using an ultrasound probe, each image being associated with positional data of the ultrasound probe relative to the vasculature at a time of image acquisition;
segmenting the plurality of two-dimensional ultrasound images considering varying acoustic signatures between one or more plaque atherosclerotic bodies and vasculature tissue;
computing a location value and an orientation value over time from positional data contained in more than one of the plurality of two-dimensional ultrasound images; and
computing a three-dimensional representation of the atherosclerotic plaque in at least one blood vessel of the vasculature from the location value, the orientation value and the segmented plurality of two-dimensional ultrasound images.

2. The method of claim 1, wherein the positional data comprises position sensors data obtained from position sensors of the ultrasound probe and wherein computing the location value and the orientation value is performed considering the position sensors data.

3. The method of claim 1, further comprising defining a three-dimensional space by analyzing images taken from one or more cameras associated with the ultrasound probe and computing the positional data for each image of the plurality of ultrasound images considering movement of the ultrasound probe relative to the three-dimensional space.

4. The method of claim 1, wherein segmenting the plurality of two-dimensional ultrasound images is performed using an artificial intelligence model trained using a dataset of preexisting images or videos segmented to distinguish plaque from surrounding vasculature tissue.

5-8. (canceled)

9. The method of claim 1, further comprising:
computing a three-dimensional reconstruction of the vasculature comprising the three-dimensional representation of the atherosclerotic plaque; and
determining, from the three-dimensional reconstruction of the vasculature, vasculature characteristics comprising one or more of:
a diameter of the vasculature;
an area of the vasculature;
a volume of the vasculature;
an intimal-medial thickness of the vasculature; and
a degree of stenosis of the vasculature.

10. (canceled)

11. The method of claim 1, further comprising generating the positional data by computing a set of spatial coordinates of an electromagnetic sensor of the ultrasound probe considering location of an electromagnetic source and at least two angular values, a timestamp being used to associate the positional data with the plurality of two-dimensional ultrasound images.

12. The method of claim 11, further comprising calibrating the electromagnetic sensor of the ultrasonic probe considering the location of the electromagnetic source.

13. The method of claim 1, further comprising:
defining, from the three-dimensional representation of the atherosclerotic plaque, plaque characteristics comprising one or more of:
an area and size of the atherosclerotic plaque;
a volume of the atherosclerotic plaque;
stenosis caused by the atherosclerotic plaque;
echogenicity of the atherosclerotic plaque;
composition of the atherosclerotic plaque;
pixel density; and
blood flow dynamics and characteristics; and
computing a risk score of vascular thromboembolic event for a subject of the two-dimensional ultrasound images, the risk being defined based at least on:
the three-dimensional representation of the atherosclerotic plaque and the three-dimensional reconstruction of the vasculature;
plaque metrics computed from at least one of the three-dimensional representation of the atherosclerotic plaque and the three-dimensional reconstruction of the vasculature, including one or more of the area and size of the atherosclerotic plaque, the volume of the atherosclerotic plaque, the stenosis caused by the atherosclerotic plaque, the echogenicity of the atherosclerotic, the plaque composition of the atherosclerotic plaque, the pixel density and the blood flow dynamics and characteristics; and
one or more of age, gender, biological sex, weight, body mass index, ethnicity, history of smoking, cholesterol level, glucose level, and history of cardiovascular events of the subject.

14-18. (canceled)

19. The method of claim 1, further comprising:
estimating a level of uncertainty associated with the computed location value and the orientation value; and
prompting a user to perform one or more scans of the vasculature using the ultrasound probe to obtain an additional set of two-dimensional ultrasound images.

20-30. (canceled)

31. A device for characterizing an atherosclerotic plaque in vasculature comprising:
one or more processors configured to:
acquire a plurality of two-dimensional ultrasound images of the vasculature using an ultrasound probe, each image being associated with positional data of the ultrasound probe relative to the vasculature at a time of image acquisition;
segment the plurality of two-dimensional ultrasound images considering varying acoustic signatures between one or more plaque atherosclerotic bodies and vasculature tissue;
compute a location value and an orientation value over time from the positional data contained in more than one of the plurality of two-dimensional ultrasound images; and
compute a three-dimensional representation of the atherosclerotic plaque in at least one blood vessel of the vasculature from the location value, the orientation value and the segmented plurality of two-dimensional ultrasound images.

32. The device of claim 31, wherein the positional data comprises position sensors data obtained from position sensors of the ultrasound probe and wherein computing the location value and the orientation value is performed considering the position sensors data.

33. The device of claim 31, wherein the one or more processors are further configured to define a three-dimensional space by analyzing images taken from one or more cameras associated with the ultrasound probe and computing the positional data for each image of the plurality of ultrasound images considering movement of the ultrasound probe relative to the three-dimensional space.

34. The device of claim 31, wherein the one or more processors are further configured to segment the plurality of two-dimensional ultrasound images by using an artificial intelligence model trained using a dataset of preexisting images or videos segmented to distinguish plaque from surrounding vasculature tissue.

35-38. (canceled)

39. The device of claim 31, wherein the one or more processors are further configured to:
compute a three-dimensional reconstruction of the vasculature comprising the three-dimensional representation of the atherosclerotic plaque; and
determine, from the three-dimensional reconstruction of the vasculature, one or more of:
a diameter of the vasculature;
an area of the vasculature;
a volume of the vasculature;
an intimal-medial thickness of the vasculature; and
a degree of stenosis of the vasculature.

40. (canceled)

41. The device of claim 31, wherein the one or more processors are further configured to generate the positional data by computing a set of spatial coordinates of an electromagnetic sensor of an ultrasound probe considering location of an electromagnetic source and at least two angular values, a timestamp being used to associate the positional data to the plurality of two-dimensional ultrasound images.

42. The device of claim 31, wherein the one or more processors are further configured to generate the positional data by further computing accelerometer data from the electromagnetic sensor.

43. The device of claim 31, wherein the one or more processors are further configured to:
define, from the three-dimensional representation of the atherosclerotic plaque, plaque characteristics comprising one or more of:
an area and size of the atherosclerotic plaque;
a volume of the atherosclerotic plaque;
stenosis caused by the atherosclerotic plaque;
echogenicity of the atherosclerotic plaque;
composition of the atherosclerotic plaque;
pixel density; and
blood flow dynamics and characteristics; and
compute a risk score of vascular thromboembolic event for a subject of the two-dimensional ultrasound images, the risk being defined based at least on:
the three-dimensional representation of the atherosclerotic plaque and the three-dimensional reconstruction of the vasculature;
plaque metrics computed from at least one of the three-dimensional representation of the atherosclerotic plaque and the three-dimensional reconstruction of the vasculature, including one or more of the area and size of the atherosclerotic plaque, the volume of the atherosclerotic plaque, the stenosis caused by the atherosclerotic plaque, the echogenicity of the atherosclerotic plaque, the plaque composition of the atherosclerotic plaque, the pixel density and the blood flow dynamics and characteristics; and
one or more of age, gender, biological sex, weight, body mass index, ethnicity, history of smoke, cholesterol level, glucose level, and history of cardiovascular events of the subject.

44. (canceled)

45. The device of claim 43, wherein the one or more processors defines the risk of vascular disease for the subject further considering published and validated thromboembolic risk score calculators, and weighs the plaque characteristics and a patient data according to contribution to thromboembolic risk score calculators.

46-48. (canceled)

49. The device of claim 31, wherein the one or more processors are further configured to:
estimate a level of uncertainty associated with the computed location value and the orientation value; and
prompt a user to perform one or more scans of the vasculature using the ultrasound probe to obtain an additional set of two-dimensional ultrasound images.

50-60. (canceled)

61. A system for characterizing an atherosclerotic plaque in vasculature comprising:
an ultrasound probe that acquires a plurality of two-dimensional ultrasound images of the vasculature;
one or more processors configured to:
associate positional data to a set of location-enhanced images of the plurality of two-dimensional ultrasound images relative to the vasculature at a time of image acquisition;
segment the location-enhanced images considering varying acoustic signatures between one or more plaque atherosclerotic bodies and vasculature tissue;
compute a location value and an orientation value over time from positional data contained in more than one of the location-enhanced images; and
compute a three-dimensional representation of the atherosclerotic plaque in at least one blood vessel of the vasculature from the location value, the orientation value and the segmented location-enhanced images.

62-86. (canceled)

87. A non-transitory computer-readable medium storing a set of instructions for characterizing an atherosclerotic plaque in vasculature, the set of instructions comprising:
one or more instructions that, when executed by one or more processors of a device, cause the device to:
acquire a plurality of two-dimensional ultrasound images of the vasculature using an ultrasound probe, each image being associated with positional data of the ultrasound probe relative to the vasculature at a time of image acquisition;
segment the plurality of two-dimensional ultrasound images considering varying acoustic signatures between one or more plaque atherosclerotic bodies and vasculature tissue;
compute a location value and an orientation value over time from positional data contained in more than one of the plurality of two-dimensional ultrasound images; and
compute a three-dimensional representation of the atherosclerotic plaque in at least one blood vessel of the vasculature from the location value, the orientation value and the segmented plurality of two-dimensional ultrasound images.

88-116. (canceled)
